SEQUENCE LISTING (58 sequences)

SEQ ID NO: 1 - protein, 119 aa, synthetic construct
  REGION 1..119: note = Amino acids
QVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSS

SEQ ID NO: 2 - protein, 107 aa, synthetic construct
  REGION 1..107: note = Amino acids
DIQMTQSPSSLSASVGDRVTITCKASQDVGTAVVWYQQKPGKAPKLLIYWASSRHEGVPDRFSGSGSGTDFTLTISSLQPEDFADYFCQQYSRYPLTFGQGTKLEIK

SEQ ID NO: 3 - protein, 119 aa, synthetic construct
  REGION 1..119: note = Amino acids
QVQLVQSGAELKKPGASVKISCKATGYTFSNYWIEWIKQAPGQGLEWIGEIFPGSGRINFNEKFKGRATFTADTSIDTTYMQLSSLTSDDSAVYYCARTKIYGNSFDYWGQGTTVTVSS

SEQ ID NO: 4 - protein, 119 aa, synthetic construct
  REGION 1..119: note = Amino acids
QVQLVQSGAEVKKPGASVKISCKATGYTFSNYWIEWIKQAPGQGLEWIGEIFPGSGRINFNEKFKGRATFTADTSIDTTYMELSRLRSDDTAVYYCARTKIYGNSFDYWGQGTLVTVSS

SEQ ID NO: 5 - protein, 119 aa, synthetic construct
  REGION 1..119: note = Amino acids
QVQLVQSGAEVKKPGASVKVSCKATGYTFSNYWIEWIKQAPGQGLEWIGEIFPGSGRINFNEKFKGRVTFTADTSISTTYMELSRLRSDDTAVYYCARTKIYGNSFDYWGQGTLVTVSS

SEQ ID NO: 6 - protein, 119 aa, synthetic construct
  REGION 1..119: note = Amino acids
QVQLVQSGAEVKKPGASVKVSCKATGYTFSNYWIEWVRQAPGQGLEWMGEIFPGSGRINFNEKFKGRVTFTADTSISTAYMELSRLRSDDTAVYYCARTKIYGNSFDYWGQGTLVTVSS

SEQ ID NO: 7 - protein, 107 aa, synthetic construct
  REGION 1..107: note = Amino acids
DIMMTQSPSFLSASVGDRVTITCKASQDVGTAVVWYQQKPGKAPKLLIYWASSRHEGVPDRFTGSGSGTDFTLTISSLQSEDFADYFCQQYSRYPLTFGQGTKLEIK

SEQ ID NO: 8 - protein, 107 aa, synthetic construct
  REGION 1..107: note = Amino acids
DIQMTQSPSSLSASVGDRVTITCRASQDVGTAVVWYQQKPGKAPKLLIYWASSRHEGVPSRFSGSGSGTDFTLTISSLQPEDFATYFCQQYSRYPLTFGQGTKLEIK

SEQ ID NO: 9 - protein, 107 aa, synthetic construct
  REGION 1..107: note = Amino acids
DIQMTQSPSSLSASVGDRVTITCRASQDVGTAVAWYQQKPGKAPKSLIYWASSRHEGVPSRFSGSGSGTDFTLTISSLQPEDFATYFCQQYSRYPLTFGQGTKLEIK

SEQ ID NO: 10 - protein, 5 aa, synthetic construct
  REGION 1..5: note = Amino acids
NYWIE

SEQ ID NO: 11 - protein, 17 aa, synthetic construct
  REGION 1..17: note = Amino acids
EIFPGSGRINFNEKFKG

SEQ ID NO: 12 - protein, 10 aa, synthetic construct
  REGION 1..10: note = Amino acids
TKIYGNSFDY

SEQ ID NO: 13 - protein, 11 aa, synthetic construct
  REGION 1..11: note = Amino acids
  VAR_SEQ 1: note = X=K or R
  VAR_SEQ 11: note = X=V or A
XASQDVGTAVX

SEQ ID NO: 14 - protein, 7 aa, synthetic construct
  REGION 1..7: note = Amino acids
WASSRHE

SEQ ID NO: 15 - protein, 9 aa, synthetic construct
  REGION 1..9: note = Amino acids
QQYSRYPLT

SEQ ID NO: 16 - protein, 119 aa, Mus musculus
QVQLQQSGAELMKPGASVKISCKATGYTFSNYWIEWIKQRPGHGLEWIGEIFPGSGRINFNEKFKGKATFTADTSSDTTYMQLSSLTSADSAVYYCARTKIYGNSFDYWGQGTTLTVSP

SEQ ID NO: 17 - protein, 107 aa, Mus musculus
DIMMTQSHKFMSTSVGDRVNITCKASQDVGTAVVWYQQKPGQSPKLLIYWASSRHNGVPDRFTGSGSGTDFTLTISNVQSEDLSDYFCQQYSRYPLTFGAGTKLELK

SEQ ID NO: 18 - protein, 30 aa, synthetic construct
  REGION 1..30: note = Amino acids
QVQLVQSGAEVKKPGASVKVSCKATGYTFS

SEQ ID NO: 19 - protein, 14 aa, synthetic construct
  REGION 1..14: note = Amino acids
WVRQAPGQGLEWIG

SEQ ID NO: 20 - protein, 32 aa, synthetic construct
  REGION 1..32: note = Amino acids
RVTFTADTSISTTYMELSRLRSDDTAVYYCAR

SEQ ID NO: 21 - protein, 11 aa, synthetic construct
  REGION 1..11: note = Amino acids
WGQGTLVTVSS

SEQ ID NO: 22 - protein, 30 aa, synthetic construct
  REGION 1..30: note = Amino acids
QVQLVQSGAELKKPGASVKISCKATGYTFS

SEQ ID NO: 23 - protein, 14 aa, synthetic construct
  REGION 1..14: note = Amino acids
WIKQAPGQGLEWIG

SEQ ID NO: 24 - protein, 32 aa, synthetic construct
  REGION 1..32: note = Amino acids
RATFTADTSIDTTYMQLSSLTSDDSAVYYCAR

SEQ ID NO: 25 - protein, 11 aa, synthetic construct
  REGION 1..11: note = Amino acids
WGQGTTVTVSS

SEQ ID NO: 26 - protein, 30 aa, synthetic construct
  REGION 1..30: note = Amino acids
QVQLVQSGAEVKKPGASVKISCKATGYTFS

SEQ ID NO: 27 - protein, 32 aa, synthetic construct
  REGION 1..32: note = Amino acids
RATFTADTSIDTTYMELSRLRSDDTAVYYCAR

SEQ ID NO: 28 - protein, 14 aa, synthetic construct
  REGION 1..14: note = Amino acids
WVRQAPGQGLEWMG

SEQ ID NO: 29 - protein, 32 aa, synthetic construct
  REGION 1..32: note = Amino acids
RVTFTADTSISTAYMELSRLRSDDTAVYYCAR

SEQ ID NO: 30 - protein, 23 aa, synthetic construct
  REGION 1..23: note = Amino acids
DIQMTQSPSSLSASVGDRVTITC

SEQ ID NO: 31 - protein, 15 aa, synthetic construct
  REGION 1..15: note = Amino acids
WYQQKPGKAPKLLIY

SEQ ID NO: 32 - protein, 32 aa, synthetic construct
  REGION 1..32: note = Amino acids
GVPDRFSGSGSGTDFTLTISSLQPEDFADYFC

SEQ ID NO: 33 - protein, 10 aa, synthetic construct
  REGION 1..10: note = Amino acids
FGQGTKLEIK

SEQ ID NO: 34 - protein, 23 aa, synthetic construct
  REGION 1..23: note = Amino acids
DIMMTQSPSFLSASVGDRVTITC

SEQ ID NO: 35 - protein, 32 aa, synthetic construct
  REGION 1..32: note = Amino acids
GVPDRFTGSGSGTDFTLTISSLQSEDFADYFC

SEQ ID NO: 36 - protein, 32 aa, synthetic construct
  REGION 1..32: note = Amino acids
GVPSRFSGSGSGTDFTLTISSLQPEDFATYFC

SEQ ID NO: 37 - protein, 15 aa, synthetic construct
  REGION 1..15: note = Amino acids
WYQQKPGKAPKSLIY

SEQ ID NO: 38 - DNA, 357 bp, synthetic construct
  misc_feature 1..357: note = Nucleic acid
  source 1..357: mol_type = other DNA
caggtgcagctggtgcagagcggcgcggaactgaaaaaaccgggcgcgagcgtgaaaattagctgcaaagcgaccggctatacctttagcaactattggattgaatggattaaacaggcgccgggccagggcctggaatggattggcgaaatttttccgggcagcggccgcattaactttaacgaaaaatttaaaggccgcgcgacctttaccgcggataccagcattgataccacctatatgcagctgagcagcctgaccagcgatgatagcgcggtgtattattgcgcgcgcaccaaaatttatggcaacagctttgattattggggccagggcaccaccgtgaccgtgagcagc

SEQ ID NO: 39 - DNA, 357 bp, synthetic construct
  misc_feature 1..357: note = Nucleic acid
  source 1..357: mol_type = other DNA
caggtgcagctggtgcagagcggcgcggaagtgaaaaaaccgggcgcgagcgtgaaaattagctgcaaagcgaccggctatacctttagcaactattggattgaatggattaaacaggcgccgggccagggcctggaatggattggcgaaatttttccgggcagcggccgcattaactttaacgaaaaatttaaaggccgcgcgacctttaccgcggataccagcattgataccacctatatggaactgagccgcctgcgcagcgatgataccgcggtgtattattgcgcgcgcaccaaaatttatggcaacagctttgattattggggccagggcaccctggtgaccgtgagcagc

SEQ ID NO: 40 - DNA, 357 bp, synthetic construct
  misc_feature 1..357: note = Nucleic acid
  source 1..357: mol_type = other DNA
caggtgcagctggtgcagagcggcgcggaagtgaaaaaaccgggcgcgagcgtgaaagtgagctgcaaagcgaccggctatacctttagcaactattggattgaatggattaaacaggcgccgggccagggcctggaatggattggcgaaatttttccgggcagcggccgcattaactttaacgaaaaatttaaaggccgcgtgacctttaccgcggataccagcattagcaccacctatatggaactgagccgcctgcgcagcgatgataccgcggtgtattattgcgcgcgcaccaaaatttatggcaacagctttgattattggggccagggcaccctggtgaccgtgagcagc

SEQ ID NO: 41 - DNA, 357 bp, synthetic construct
  misc_feature 1..357: note = Nucleic acid
  source 1..357: mol_type = other DNA
caggtgcagctggtgcagagcggcgcggaagtgaaaaaaccgggcgcgagcgtgaaagtgagctgcaaagcgaccggctatacctttagcaactattggattgaatgggtgcgccaggcgccgggccagggcctggaatggattggcgaaatttttccgggcagcggccgcattaactttaacgaaaaatttaaaggccgcgtgacctttaccgcggataccagcattagcaccacctatatggaactgagccgcctgcgcagcgatgataccgcggtgtattattgcgcgcgcaccaaaatttatggcaacagctttgattattggggccagggcaccctggtgaccgtgagcagc

SEQ ID NO: 42 - DNA, 357 bp, synthetic construct
  misc_feature 1..357: note = Nucleic acid
  source 1..357: mol_type = other DNA
caggtgcagctggtgcagagcggcgcggaagtgaaaaaaccgggcgcgagcgtgaaagtgagctgcaaagcgaccggctatacctttagcaactattggattgaatgggtgcgccaggcgccgggccagggcctggaatggatgggcgaaatttttccgggcagcggccgcattaactttaacgaaaaatttaaaggccgcgtgacctttaccgcggataccagcattagcaccgcgtatatggaactgagccgcctgcgcagcgatgataccgcggtgtattattgcgcgcgcaccaaaatttatggcaacagctttgattattggggccagggcaccctggtgaccgtgagcagc

SEQ ID NO: 43 - DNA, 321 bp, synthetic construct
  misc_feature 1..321: note = Nucleic acid
  source 1..321: mol_type = other DNA
gatattatgatgacccagagcccgagctttctgagcgcgagcgtgggcgatcgcgtgaccattacctgcaaagcgagccaggatgtgggcaccgcggtggtgtggtatcagcagaaaccgggcaaagcgccgaaactgctgatttattgggcgagcagccgccatgaaggcgtgccggatcgctttaccggcagcggcagcggcaccgattttaccctgaccattagcagcctgcagagcgaagattttgcggattatttttgccagcagtatagccgctatccgctgacctttggccagggcaccaaactggaaattaaa

SEQ ID NO: 44 - DNA, 321 bp, synthetic construct
  misc_feature 1..321: note = Nucleic acid
  source 1..321: mol_type = other DNA
gatattcagatgacccagagcccgagcagcctgagcgcgagcgtgggcgatcgcgtgaccattacctgcaaagcgagccaggatgtgggcaccgcggtggtgtggtatcagcagaaaccgggcaaagcgccgaaactgctgatttattgggcgagcagccgccatgaaggcgtgccggatcgctttagcggcagcggcagcggcaccgattttaccctgaccattagcagcctgcagccggaagattttgcggattatttttgccagcagtatagccgctatccgctgacctttggccagggcaccaaactggaaattaaa

SEQ ID NO: 45 - DNA, 321 bp, synthetic construct
  misc_feature 1..321: note = Nucleic acid
  source 1..321: mol_type = other DNA
gatattcagatgacccagagcccgagcagcctgagcgcgagcgtgggcgatcgcgtgaccattacctgccgcgcgagccaggatgtgggcaccgcggtggtgtggtatcagcagaaaccgggcaaagcgccgaaactgctgatttattgggcgagcagccgccatgaaggcgtgccgagccgctttagcggcagcggcagcggcaccgattttaccctgaccattagcagcctgcagccggaagattttgcgacctatttttgccagcagtatagccgctatccgctgacctttggccagggcaccaaactggaaattaaa

SEQ ID NO: 46 - DNA, 321 bp, synthetic construct
  misc_feature 1..321: note = Nucleic acid
  source 1..321: mol_type = other DNA
gatattcagatgacccagagcccgagcagcctgagcgcgagcgtgggcgatcgcgtgaccattacctgccgcgcgagccaggatgtgggcaccgcggtggcgtggtatcagcagaaaccgggcaaagcgccgaaaagcctgatttattgggcgagcagccgccatgaaggcgtgccgagccgctttagcggcagcggcagcggcaccgattttaccctgaccattagcagcctgcagccggaagattttgcgacctatttttgccagcagtatagccgctatccgctgacctttggccagggcaccaaactggaaattaaa

SEQ ID NO: 47 - protein, 119 aa, synthetic construct
  REGION 1..119: note = Amino acids
  VAR_SEQ 11: note = X=L or V
  VAR_SEQ 20: note = X= I or V
  VAR_SEQ 37: note = X=I or V
  VAR_SEQ 38: note = X=K or R
  VAR_SEQ 48: note = X=I or M
  VAR_SEQ 68: note = X=A or V
  VAR_SEQ 77: note = X=D or S
  VAR_SEQ 79: note = X=T or A
  VAR_SEQ 82: note = X=Q or E
  VAR_SEQ 85: note = X=S or R
  VAR_SEQ 87: note = X=T or R
  VAR_SEQ 91: note = X= S or T
  VAR_SEQ 114: note = X=T or L
QVQLVQSGAEXKKPGASVKXSCKATGYTFSNYWIEWXXQAPGQGLEWXGEIFPGSGRINFNEKFKGRXTFTADTSIXTXYMXLSXLXSDDXAVYYCARTKIYGNSFDYWGQGTXVTVSS

SEQ ID NO: 48 - protein, 107 aa, synthetic construct
  REGION 1..107: note = Amino acids
  VAR_SEQ 3: note = X=M or Q
  VAR_SEQ 10: note = X=F or S
  VAR_SEQ 24: note = X=K or R
  VAR_SEQ 34: note = X=V or A
  VAR_SEQ 46: note = X=L or S
  VAR_SEQ 60: note = X=D or S
  VAR_SEQ 63: note = X=T or S
  VAR_SEQ 80: note = X=S or P
  VAR_SEQ 85: note = X=D or T
DIXMTQSPSXLSASVGDRVTITCXASQDVGTAVXWYQQKPGKAPKXLIYWASSRHEGVPXRFXGSGSGTDFTLTISSLQXEDFAXYFCQQYSRYPLTFGQGTKLEIK

SEQ ID NO: 49 - protein, 236 aa, synthetic construct
  REGION 1..236: note = Amino acids
MDMRVPAQLLGLLLLWLRGARCDIQMTQSPSSLSASVGDRVTITCKASQDVGTAVVWYQQKPGKAPKLLIYWASSRHEGVPDRFSGSGSGTDFTLTISSLQPEDFADYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 446 aa, synthetic construct
  REGION 1..446: note = Amino acids
QVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 449 aa, synthetic construct
  REGION 1..449: note = Amino acids
QVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 711 bp, synthetic construct
  misc_feature 1..711: note = Nucleic acid
  source 1..711: mol_type = other DNA
atggacatgagggtccccgctcagctcctggggctcctgctactctggctccgaggtgccagatgtgacatccagatgacccagtccccttcctctttatccgcttccgtgggcgatagggtgaccatcacttgtaaggcctcccaagatgtgggcacagctgtggtgtggtaccagcagaagcccggcaaggcccccaagctgctgatctactgggcttcctctcgtcacgagggcgtgcccgatcgtttctccggctccggatccggcaccgacttcactttaaccatctcctctttacagcccgaggacttcgccgactacttctgccagcagtactctcgttaccctttaacctttggccaaggtaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 53 - DNA, 718 bp, synthetic construct
  misc_feature 1..718: note = Nucleic acid
  source 1..718: mol_type = other DNA
atggacatgagagtgcctgctcagctgctgggactgctgctgttgtggttgagaggcgccagatgcgacatccagatgacccagtctccatcctctctgtccgcctctgtgggcgacagagtgaccatcacatgcaaggcctctcaggatgtgggcaccgccgttgtgtggtatcagcagaagcctggcaaggcccctaagctgctgatctactgggcctcctctagacacgagggcgtgcccgatagattctccggctctggctctggcaccgactttaccctgacaatctccagcctgcagcctgaggacttcgccgactacttctgccagcagtacagcagataccctctgacctttggccagggcaccaagctggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctgatgaattc

SEQ ID NO: 54 - DNA, 1398 bp, synthetic construct
  misc_feature 1..1398: note = Nucleic acid
  source 1..1398: mol_type = other DNA
atggggtcaaccgccatccttggcctcctcctggctgttctccaaggagtctgtgcccaagttcagctggtgcagagcggcgctgaggtgaagaagcccggtgcctccgtgaaggtgtcttgtaaggccaccggctacaccttctccaactactggatcgagtgggtgaggcaagctcccggtcaaggtttagagtggatcggagagatcttccccggctccggccggatcaacttcaacgagaagttcaagggccgggtgacctttaccgccgacaccagcatctccaccacctacatggagctgtctcgtctgaggtccgacgacaccgccgtgtactactgcgctcgtaccaagatctacggcaactccttcgactactggggccaaggtactttagtgacagtgtcctccgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 55 - DNA, 1402 bp, synthetic construct
  misc_feature 1..1402: note = Nucleic acid
  source 1..1402: mol_type = other DNA
atgggctctacagctatcctgggactgctgctggctgtgctgcaaggcgtttgtgctcaggtgcagctggttcagtctggcgccgaagtgaagaaacctggcgcctctgtgaaggtgtcctgcaaggctaccggctacaccttctccaactactggatcgagtgggtccgacaggctcctggacaaggcctggaatggatcggcgagatctttcctggcagcggccggatcaacttcaacgagaagttcaagggcagagtgaccttcaccgccgacacctccatctccaccacctacatggaactgtcccggctgagatctgacgacaccgccgtgtactactgcgcccggaccaagatctacggcaactccttcgattactggggccagggcacactggtcaccgtgtcctctgcttctacaaagggcccaagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggctgatgaattc

SEQ ID NO: 56 - protein, 260 aa, synthetic construct
  REGION 1..260: note = Amino acids
MGWSCIILFLVATATGVHSQVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSSGGGGSGGGGSGGGGSDIQMTQSPSSLSASVGDRVTITCKASQDVGTAVVWYQQKPGKAPKLLIYWASSRHEGVPDRFSGSGSGTDFTLTISSLQPEDFADYFCQQYSRYPLTFGQGTKLEIK

SEQ ID NO: 57 - protein, 260 aa, synthetic construct
  REGION 1..260: note = Amino acids
MGWSCIILFLVATATGVHSQVQLVQSGAEVKKPGASVKVSCKATGYTFSNYWIEWIKQAPGQGLEWIGEIFPGSGRINFNEKFKGRVTFTADTSISTTYMELSRLRSDDTAVYYCARTKIYGNSFDYWGQGTLVTVSSGGGGSGGGGSGGGGSDIQMTQSPSSLSASVGDRVTITCRASQDVGTAVAWYQQKPGKAPKSLIYWASSRHEGVPSRFSGSGSGTDFTLTISSLQPEDFATYFCQQYSRYPLTFGQGTKLEIK

SEQ ID NO: 58 - protein, 10 aa, synthetic construct
  REGION 1..10: note = Amino acids
GYTFSNYWIE

(19) United States
(12) Patent Application Publication
(10) Pub. No.: US 2026/0152568 A1
(43) Pub. Date: Jun. 4, 2026

(54) ANTIBODIES AGAINST THE POLIOVIRUS RECEPTOR (PVR) AND USES THEREOF

(71) Applicant: NECTIN THERAPEUTICS LTD., Jerusalem (IL)

(72) Inventors: Philipp ALBERTS, Amsterdam (NL); Arif JETHA, Toronto (CA); Johan FRANSSON, San Diego, CA (US); Yazen JMEIAN, New Haven, CT (US); Joanne HULME, Toronto (CA); Pinchas TSUKERMAN, Jerusalem (IL)

(73) Assignee: NECTIN THERAPEUTICS LTD., Jerusalem (IL)

(21) Appl. No.: 19/450,115

(22) Filed: Jan. 15, 2026

Related U.S. Application Data
(63) Continuation of application No. 17/764,373, now Pat. No. 12,570,757.
(60) Provisional application No. 62/912,534, filed on Oct. 8, 2019.

(51) Int. Cl. C07K 16/28; A61K 39/00; A61P 31/14; A61P 35/02; A61P 37/02

(52) U.S. Cl. CPC C07K 16/2896; A61P 31/14; A61P 35/02; A61P 37/02; A61K 2039/505; C07K 2317/24; C07K 2317/30; C07K 2317/565; C07K 2317/622; C07K 2317/76; C07K 2317/92

(57) ABSTRACT

The present application provides humanized antibodies and antigen binding fragments thereof that bind to human poliovirus (PVR). The antibodies are useful in the treatment of tumors or cancers. The present application also provides a method of treating a cancer in an individual afflicted with a cancer comprising administering to the individual a therapeutically effective amount of chimeric antigen receptor (CAR) NK or T cells.

### CROSS-REFERENCE TO RELATED APPLICATIONS

This application is a continuation of U.S. patent application Ser. No. 17/764,373, filed Mar. 28, 2022, which is a 35 U.S.C. § 371 National Stage of International Patent Application No. PCT/IL2020/051082, filed Oct. 7, 2020, claiming benefit from U.S. Provisional Patent Application No. 62/912,534, filed Oct. 8, 2019, the disclosures of which are incorporated herein in their entirety by reference, and priority is claimed to each of the foregoing.

### INCORPORATION OF SEQUENCE LISTING

The instant application contains a Sequence Listing which has been submitted electronically in XML format and is hereby incorporated by reference in its entirety. Said XML copy, created on Feb. 15, 2026, is named “NECTIN001 PCT Sequence listing ST26_Corrected.xml” and is 91,444 bytes in size.

### FIELD OF THE INVENTION

The present invention is in the field of immunotherapy and relates to humanized antibodies, comprising specific sets of CDR and framework sequences that specifically bind to human poliovirus receptor (PVR). Pharmaceutical compositions comprising these humanized antibodies and their uses are also included.

### BACKGROUND OF THE INVENTION

Poliovirus receptor (PVR), also termed CD155, is a transmembrane glycoprotein involved in mediating cell adhesion to extracellular matrix molecules. It was previously described as a tumor antigen and as a potential target for therapeutic intervention as its expression is up-regulated in neuroectodermal cancers, including glioblastoma multiforme, medulloblastoma, and colorectal carcinoma (Solecki et al., J. Biol. Chem. 2002, 277: 25697-700), as well as in pancreatic cancer (Nishiwada et al., Anticancer Res. 2015, 35(4): 2287-97). PVR is also known to enhance the serum-induced activation of the Ras-Raf-MEK-ERK signaling, up-regulating cyclins D2 and E, and down-regulated p27Kip1, eventually shortening the period of the G0/G1 phase of the cell cycle (Kakunaga 2004, J. Biological Chemistry, 279, 36419-36425. For that reason, blocking of PVR on tumor cells is anticipated to reduce their viability. PVR has also a critical role in angiogenesis and is suggested to regulate the VEGF-induced angiogenesis by controlling the interaction of Vascular endothelial growth factor receptor 2 (VEGFR2) with integrin a(v)B(3), and the VEGFR2-mediated Rap1-Akt signaling pathway (Kinugasa et al., 2012, Circ Res. 2012, 110(5),716-26). Additionally, PVR is complexing with IGF1R and participating in tyrosine-protein kinase Met (cMet) signaling and blocking the complex formation reduced cell viability and angiogenesis (Lee et al., Scientific Reports 2014, 20, 4, 7139).

In recent years it became evident that PVR is a critical immune check point ligand (Brilc P. K. et al 2019 Cell Mol Immunology). PVR expression is upregulated in both malignant cells and tumor-infiltrating myeloid cells in humans and mice. PVR−/− mice display reduced tumor growth and metastasis via DNAM-1 (CD226) upregulation and enhanced effector function of CD8+ T and NK cells, respectively. Blockade of Programmed cell death protein 1 (PD-1) or both PD-1 and cytotoxic T-lymphocyte-associated protein 4 (CTLA4) is more effective in settings in which PVR was limiting, suggesting the clinical potential of combinatory therapy using PD-1/PD-L1 and PVR blockade (LI X. Y et. al JCI 2018). Moreover, in clinical settings, the expression of PD-L1 and PVR is independently regulated, which allowed stratification of patients who were treated with anti-PD-1 antibody into 4 groups according to the expression levels of PD-L1 and PVR. High PVR expression in PD-L1-low-expressing patients enriched non-responders. This was further validated using a genetically engineered cancer model. These findings bolster the significance of PVR as a critical immune check point in tumor immune-therapy (Lee B. R et al JCI. Insight 2020). PVR involvement in metastasis was demonstrated by injecting cancer cells to the tail of mice and measuring metastasis to the lungs. It has been shown that the upregulated PVR in cancer cells transinteracts with its counter-receptor in platelets, and that this trans-interaction enhances the metastasis of the cancer cells to the lungs (Morimoto et al., Oncogene (2008) 27, 264-273).

WO2017149538 to one of the present inventors discloses murine antibodies and fragments thereof which bind to PVR as well as encoding polynucleotide sequences and hybridoma cells producing these antibodies.

U.S. Patent Application No. 20070041985 discloses molecules specifically binding to at least one intra- or extracellular domain of the PVR, wherein the molecule has the ability to modulate a receptor mediated adhesion, trafficking and/or invasion behavior of a cell expressing the PVR or any derivative thereof.

U.S. Patent Application No. 20090215175 provides molecules (e.g. small chemical compounds, oligonucleotides, polypeptides, antibodies, and antibody fragments) which modulate the PVR functions necessary for adhesion, trafficking, invasion and/or metastatic potential of cells. The molecules can be used for the treatment of cells having a metastatic potential, metastasis and cancer.

There is an unmet need to provide humanized antibodies recognizing human PVR which are safer and more potent and can be used diagnostically and therapeutically in diseases involving PVR expression.

### SUMMARY OF THE INVENTION

Described herein, according to some embodiments, are humanized antibodies that specifically bind human poliovirus receptor (PVR; CD155) and prevent binding of PVR to at least one of ligands, T cell immunoreceptor with Ig and ITIM domains (TIGIT), CD96 and CD226 (DNAM-1). The humanized antibodies of the present invention, selected from a larger collection of antibody clones, have improved properties compared to other known anti-PVR antibodies. These improved properties include but are not limited to reduced immunogenicity potential, improved binding affinity and activity, biophysical properties and improved expression. As PVR binding to CD226 results in down-regulation of surface expression of CD226 on T and NK cells and reduced activity of CD226 to stimulate T and NK cells and tumor cell killing, the antibodies of the present invention can restore the expression and/or activity of CD226 on these cells. Proper expression and functioning of CD226 allows for increased tumor killing by immune cells, especially CD8+ T cells and NK cells.

A large collection of humanized antibodies was produced by combining specific sets of CDR sequences and human framework sequences and introducing specific mutations in these sequences to produce improved antibodies with modified variable regions. The newly designed humanized variable regions preserve the residues critical for the maintenance of the antibody's conformation and binding affinity, while having the lowest incidence of potential T cell epitopes, thus minimizing the risk of adverse immune response towards the antibodies. The antibodies disclosed herein were designed based on factors including homology, T-cell epitopes, key residues, and predicted structures.

Unexpectedly, variants having a combination of specific human frameworks a point mutation of Glutamic acid to Asparagine in the last residue of CDR2 of the light chain variable region (position 56 according to Kabat numbering), show strong affinity to human PVR and improved immune activity.

Several humanized antibody variants according to the invention were found to be particularly suitable for chimeric antigen receptor (CAR) applications due to their lower affinity, which may be useful in targeting the highly expressed PVR tumor cells, without targeting normal tissues.

Advantageously, several humanized antibody variants according to the present invention have improved producibility and are capable of being produced with exceptionally high yield compared to other variants.

It is now disclosed that the humanized antibody described herein show high efficacy in cytotoxic T and NK cell stimulation, and in treatment of cancer in humanized mouse models, including in vivo models of pancreatic cancer and lung cancer.

The present invention thus provides, in some embodiments, highly-specific, non-immunogenic, humanized antibodies against human PVR having improved affinity, activity and/or reproducibility.

The present invention provides, according to one aspect, a humanized antibody that specifically binds human poliovirus receptor (PVR, CD155), or a fragment thereof comprising at least the antigen binding site, wherein the antibody or a fragment thereof comprises a heavy chain and a light chain, wherein the heavy chain comprises a variable region having an amino acid sequence at least about 90% identical to a sequence selected from the group consisting of SEQ ID NO: 1, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 2, SEQ ID NO: 7, SEQ ID NO: 8 and SEQ ID NO: 9.

According to some embodiments, the antibody comprises heavy-chain variable region amino-acid sequence comprising the CDR sequences set forth in SEQ ID NO:10, SEQ ID NO:11 and SEQ ID NO:12, and light-chain variable region amino-acid sequence comprising the CDR sequences set forth in SEQ ID NO:13, SEQ ID NO:14 and SEQ ID NO:15. According to some embodiments, the humanized antibody or fragment thereof is an IgG monoclonal antibody. According to some embodiments, the humanized monoclonal antibody has a heavy chain constant region selected from IgG4 and IgG1. In certain embodiments, the humanized antibody or fragment thereof is an IgG4 subclass. In certain embodiments, the humanized antibody or antigen binding fragment thereof is an IgG1 subclass.

According to some embodiments, the humanized antibody or fragment thereof comprises a human IgG4 constant region having S228P (also named S241P) substitution in the hinge region.

According to some embodiments, the humanized antibody or fragment thereof is a monoclonal antibody, Fab, F(ab)₂, a single-domain antibody, or a single chain variable fragment (scFv).

According to some embodiments, the humanized antibody or antigen binding fragment thereof comprises a heavy chain variable region comprising the amino acid sequence QVQLVQSGAE(L/V)KKPGASVK(I/V)SCKATGYTFSNYWIEW(I/V)(K/R)QAPGQGLE W(I/M)GEIFPGSGRINFNEKFKGR(A/V)TFTADTSI(D/S)T(T/A)YM(Q/E)LS(S/R)L(T/R) SDD(S/T)AVYYCARTKIYGNSFDYWGQGT(T/L)VTVSS (SEQ ID NO: 47); and a light chain variable region comprising the amino acid sequence DI(M/Q)MTQSPS(F/S)LSASVGDRVTITC(K/R)ASQDVGTAV(V/A)WYQQKPGKAPK(L/S)LIYWASSRHEGVP(D/S)RF(T/S)GSGSGTDFTLTISS LQ(S/P)EDFA(D/T)YFCQQYSRYPLTFGQGT KLEIK (SEQ ID NO: 48).

According to some embodiments, the humanized antibody or fragment thereof comprises a heavy chain variable region comprising:
i. a set of three CDR sequences comprising the sequences set forth in SEQ ID Nos. 10-12; and
ii. a set of four heavy chain framework (FR) sequences: (A) FR-H1 selected from the group consisting of SEQ ID NOs: 18, 22, and 26; (B) FR-H2 selected from the group consisting of SEQ ID NOs: 19, 23, and 28; (C) FR-H3 selected from the group consisting of SEQ ID NOs: 20, 24, 27, and 29; and (D) FR-H4 selected from the group consisting of SEQ ID NOs: 21 and 25.

According to some embodiments, the humanized antibody or antigen binding fragment thereof comprising a light chain variable region comprising:
i. a set of three CDR sequences comprising the sequences set forth in SEQ ID Nos. 13-15; and
ii. a set of four light chain framework sequences: (A) FR-L1 selected from the group consisting of SEQ ID NOs: 30 and 34; (B) FR-L2 selected from the group consisting of SEQ ID NOs: 31 and 37; (C) FR-L3 selected from the group consisting of SEQ ID NOs: 32, 35, and 36; and (D) FR-L4 is SEQ ID NO: 33.

According to some embodiments, the humanized antibody or antigen binding fragment thereof comprising a heavy chain variable region and a light chain variable region, the heavy chain variable region comprising:
i. a set of three CDR sequences comprising the sequences set forth in SEQ ID Nos. 10-12; and
ii. a set of four heavy chain (HC) framework (FR) sequences: (A) FR-H1 selected from the group consisting of SEQ ID NOs: 18, 22, and 26; (B) FR-H2 selected from the group consisting of SEQ ID NOs: 19, 23, and 28; (C) FR-H3 selected from the group consisting of SEQ ID NOs: 20, 24, 27, and 29; (D) FR-H4 selected from the group consisting of SEQ ID NOs: 21 and 25;
and the light chain variable region comprising:
i. a set of three CDR sequences comprising the sequences set forth in SEQ ID Nos. 13-15; and
ii. a set of four light chain (LC) framework (FR) sequences: (A) FR-L1 selected from the group consisting of SEQ ID NOs: 30 and 34; (B) FR-L2 selected from the group consisting of SEQ ID NOs: 31 and 37; (C) FR-L3 selected from the group consisting of SEQ ID NOs: 32, 35, and 36; and (D) FR-L4 is SEQ ID NO: 33.

According to some embodiments, the heavy chain variable region of the humanized monoclonal antibody comprises an amino acid sequence at least about 90% identical to a sequence selected from the group consisting of SEQ ID NO: 1, SEQ ID NO: 3, SEQ ID NO: 4, SEQ ID NO: 5 and SEQ ID NO: 6; and the light chain variable region comprises an amino acid sequence at least about 90% identical to a sequence selected from the group consisting of SEQ ID NO: 2, SEQ ID NO: 7, SEQ ID NO: 8 and SEQ ID NO: 9. According to some embodiments, the heavy chain variable region of the humanized monoclonal antibody comprises an amino acid sequence at least about 95% identical to a sequence selected from the group consisting of SEQ ID NO: 1, SEQ ID NO: 3, SEQ ID NO: 4, SEQ ID NO: 5 and SEQ ID NO: 6; and the light chain variable region comprises an amino acid sequence at least about 95% identical to a sequence selected from the group consisting of SEQ ID NO: 2, SEQ ID NO: 7, SEQ ID NO: 8 and SEQ ID NO: 9. In certain embodiments, the heavy chain variable region of the humanized monoclonal antibody comprises an amino acid sequence at least about 97% identical to a sequence selected from the group consisting of SEQ ID NO: 1, SEQ ID NO: 3, SEQ ID NO: 4, SEQ ID NO: 5 and SEQ ID NO: 6, and the light chain variable region comprises an amino acid sequence at least about 97% identical to a sequence selected from the group consisting of SEQ ID NO: 2, SEQ ID NO: 7, SEQ ID NO: 8 and SEQ ID NO: 9. In certain embodiments, the heavy chain variable region of the humanized monoclonal antibody comprises an amino acid sequence selected from the group consisting of SEQ ID NO: 1, SEQ ID NO: 3, SEQ ID NO: 4, SEQ ID NO: 5 and SEQ ID NO: 6, and the light chain variable region comprises an amino acid sequence selected from the group consisting of SEQ ID NO: 2, SEQ ID NO: 7, SEQ ID NO: 8 and SEQ ID NO: 9.

According to some embodiments, the humanized antibody comprises a combination of a heavy chain variable region and a light chain variable region, wherein the combination is selected from the group consisting of:
i. a heavy chain variable region sequence set forth in SEQ ID NO: 1 and a light chain variable region sequence set forth in SEQ ID NO: 2;
ii. a heavy chain variable region sequence set forth in SEQ ID NO: 4 and a light chain variable region sequence set forth in SEQ ID NO: 8;
iii. a heavy chain variable region sequence set forth in SEQ ID NO: 5 and a light chain variable region sequence set forth in SEQ ID NO: 2;
iv. a heavy chain variable region sequence set forth in SEQ ID NO: 5 and a light chain variable region sequence set forth in SEQ ID NO: 8;
v. a heavy chain variable region sequence set forth in SEQ ID NO: 4 and a light chain variable region sequence set forth in SEQ ID NO: 2;
vi. a heavy chain variable region sequence set forth in SEQ ID NO: 1 and a light chain variable region sequence set forth in SEQ ID NO: 8;
vii. a heavy chain variable region sequence set forth in SEQ ID NO: 6 and a light chain sequence set forth in SEQ ID NO: 2; and
viii. a heavy chain variable region sequence set forth in SEQ ID NO: 6 and a light chain variable region sequence set forth in SEQ ID NO: 8.

According to some embodiments, the heavy chain variable region of the humanized monoclonal antibody comprises an amino acid sequence at least about 90% identical to that set forth in SEQ ID NO: 1, and the light chain variable region comprises an amino acid sequence at least about 90% identical to that set forth in SEQ ID NO: 2.

According to some embodiments, the heavy chain variable region of the humanized monoclonal antibody comprises an amino acid sequence identical to that set forth in SEQ ID NO: 1, and the light chain variable region comprises an amino acid sequence identical to that set forth in SEQ ID NO: 2.

According to some embodiments, the humanized antibody inhibits binding of PVR to at least one of TIGIT, CD96, and CD226.

According to some embodiments, the antibody inhibits binding of PVR to TIGIT, CD96, and CD226.

According to some embodiments, the humanized antibody is an IgG4 antibody comprising a heavy chain sequences set forth in SEQ ID NO: 49, or a sequence having at least 90% identity. According to some embodiments, the humanized antibody is an IgG1 comprising a heavy chain sequence set forth in SEQ ID NO: 50 or a sequence having at least 90% identity.

According to some embodiments, the humanized antibody comprising a light chain sequence set forth in SEQ ID NO: 49.

According to some embodiments, the humanized antibody exhibits improved antibody-dependent cellular cytotoxicity (ADCC) or complement dependent cytotoxicity (CDC) compared to other, known antibodies.

Polynucleotide sequences encoding the humanized antibody or antigen binding fragment thereof, are provided according to another aspect of the invention.

According to some embodiments, a polynucleotide sequence encoding the amino acid sequences of a heavy chain variable region, a light chain variable region or both, as described above is provided.

According to some embodiments, a polynucleotide is provided encoding a heavy chain variable region comprising an amino acid sequence selected from the group consisting of SEQ ID NO: 1, SEQ ID NO: 3, SEQ ID NO: 4, SEQ ID NO: 5 and SEQ ID NO: 6.

According to some embodiments, a polynucleotide is provided encoding a light chain variable region comprising an amino acid sequence selected from the group consisting of SEQ ID NO: 2, SEQ ID NO: 7, SEQ ID NO: 8 and SEQ ID NO: 9.

According to some embodiments, the polynucleotide encodes a humanized antibody or antibody fragment thereof comprising: a heavy chain variable region comprising an amino acid sequence selected from the group consisting of SEQ ID NO: 1, SEQ ID NO: 3, SEQ ID NO: 4, SEQ ID NO: 5 and SEQ ID NO: 6; and a light chain variable region comprising an amino acid sequence selected from the group consisting of SEQ ID NO: 2, SEQ ID NO: 7, SEQ ID NO: 8 and SEQ ID NO: 9. Each combination of heavy and light chain variable regions represents a separate embodiment of the invention.

According to some embodiments, the polynucleotide sequence encoding the humanized antibody heavy chain variable region comprises a sequence selected from the group consisting of SEQ ID NOs: 38-42, or a variant thereof having at least 90% sequence identity. Each possibility represents a separate embodiment of the invention.

According to some embodiments, the polynucleotide sequence encoding the humanized antibody light chain variable region comprises a sequence selected from the group consisting of SEQ ID NOs: 43-46, or a variant thereof having at least 90% sequence identity. Each possibility represents a separate embodiment of the invention.

In a further aspect, the present invention provides a nucleic acid construct comprising a nucleic acid molecule encoding at least one humanized antibody chain or fragment thereof as described herein. According to some embodiments the nucleic acid construct is a plasmid.

Also described is a cell line comprising the nucleic acids encoding the antibodies of the present invention. The cell line is for expression of the humanized antibody or fragment thereof as described herein. In certain embodiments, the cell line is a mammalian cell line such as a Chinese Hamster Ovary (CHO) cell line.

According to some embodiments, the cell line is a bacterial, plant, murine (e.g., NS0 and Sp2/0), rat (e.g., YB2/0), hamster (e.g., BHK and CHO) or human (e.g., PER.C6).

According to an aspect, the present invention provides a chimeric antigen receptor (CAR) comprising an extracellular portion (binding domain), containing any of the humanized antibodies or fragment thereof as described herein. According to some embodiments, a CAR comprising a combination of heavy and light chain variable region sequences described above, having unique combination of CDR and framework sequences and improved binding and other properties, is provided.

According to some embodiments, the CAR comprises a combination of heavy and light chain variable regions, the heavy chain variable region comprises an amino acid sequence with at least 90% sequence identity to a sequence selected from the group consisting of SEQ ID NO: 1, SEQ ID NO: 3, SEQ ID NO: 4, SEQ ID NO: 5 and SEQ ID NO: 6; and the light chain variable region comprises an amino acid sequence with at least 90% sequence identity to a sequence selected from the group consisting of SEQ ID NO: 2, SEQ ID NO: 7, SEQ ID NO: 8 and SEQ ID NO: 9.

According to some embodiments, the CAR comprises a heavy chain variable region comprising an amino acid sequence selected from the group consisting of SEQ ID NO: 1, SEQ ID NO: 3, SEQ ID NO: 4, SEQ ID NO: 5 and SEQ ID NO: 6, and a light chain variable region comprising an amino acid sequence selected from the group consisting of SEQ ID NO: 2, SEQ ID NO: 7, SEQ ID NO: 8 and SEQ ID NO: 9.

According to some embodiments, the CAR comprises a combination of a humanized antibody heavy and light chain variable regions, wherein the combination is selected from the group consisting of:
i. a heavy chain variable region sequence set forth in SEQ ID NO: 1 and a light chain variable region sequence set forth in SEQ ID NO: 9;
ii. a heavy chain variable region sequence set forth in SEQ ID NO: 3 and a light chain variable region sequence set forth in SEQ ID NO: 9;
iii. a heavy chain variable region sequence set forth in SEQ ID NO: 4 and a light chain variable region sequence set forth in SEQ ID NO: 9;
iv. a heavy chain variable region sequence set forth in SEQ ID NO: 5 and a light chain variable region sequence set forth in SEQ ID NO: 9; and
v. a heavy chain variable region sequence set forth in SEQ ID NO: 6 and a light chain variable region sequence set forth in SEQ ID NO: 9.

According to some embodiments, the CAR comprises a heavy chain variable region sequence selected from the group consisting of SEQ ID NO: 1, 3, 4, 5, and 6, and a light chain variable region sequence set forth in SEQ ID NO: 9, a transmembrane domain, and an intracellular T cell signaling domain.

A single chain variable region (scFv) comprising the heavy chain and light chain variable regions of the antibodies described herein is also provided according to the present invention. According to certain embodiments, there is a hinge region between the variable regions.

According to some embodiments, the amino acid sequence of the scFv is set forth in a sequence selected from SEQ ID NO: 56, SEQ ID NO: 57, and an analog thereof having at least 90% sequence similarity to any of said sequences.

According to some embodiments, the CAR comprises an amnio acid sequences set forth in any one of SEQ ID NO: 56 and SEQ ID NO: 57.

According to some embodiments, the CAR comprises a scFv sequence and at least one protein domain selected from the group consisting of a CD8 Stalk domain, a CD28 TM domain, a 41BB domain, and a CD3((CD3Z, Zetta) domain. According to some embodiments, the CAR comprises a CD8 Stalk domain. According to some embodiments, the CAR comprises a CD28 TM domain. According to some embodiments, the CAR comprises a CD3Z domain.

According to some embodiments, the CAR comprises a 41BB domain. According to specific embodiments, the CAR comprises a CD8 Stalk domain, a CD28 TM domain, a 41BB domain, and a CD3Z domain.

According to some embodiments, the CAR comprises a scFv sequence comprising the PVR binding site of any antibody disclosed above and at least one domain selected from the group consisting of: CD8 Stalk domain, a CD28 TM domain, a 41BB domain, and a CD3Z domain. According to specific embodiments, the CAR comprises a scFv sequence comprising the PVR binding site of any antibody disclosed above and a CD8 Stalk domain, a CD28 TM domain, a 41BB domain, and a CD3Z domain.

According to some embodiments, a lymphocyte engineered to express the CAR described herein is provided. According to some embodiments, a T cell engineered to express the CAR described herein is provided. According to additional embodiments, an NK cell engineered to express the CAR described herein is provided.

According to specific embodiments, an engineered T cell is provided, expressing a scFv sequence selected from the group consisting of: SEQ ID NO: 56, SEQ ID NO: 57, or an analog thereof having at least 90% sequence similarity to any of said sequences; a CD8 Stalk domain, a CD28 TM domain, a 41BB domain, and a CD3Z domain.

According to an aspect, the present invention provides a method of treating cancer in a subject comprising administering a therapeutically effective amount of at least one lymphocyte comprising the CAR as described herein to said subject.

The present invention provides, according to another aspect, a pharmaceutical composition comprising the humanized antibody or antigen binding fragment described herein and a pharmaceutically acceptable excipient, carrier, or diluent.

Any administration mode may be used to deliver the compositions of the present invention to a subject in need thereof, including parenteral and enteral administration modes.

According to some embodiments, the pharmaceutical composition is formulated for injection or infusion. According to some embodiments, the pharmaceutical composition is formulated for intravenous administration. In certain embodiments, the pharmaceutical composition is formulated for intratumoral administration.

According to some embodiments, the humanized antibody or antigen binding fragment thereof or the pharmaceutical composition is for use in increasing surface expression and/or signaling of CD226 on CD8+ and CD4+ T cells.

According to embodiments, the humanized antibody or antigen binding fragment thereof, or the pharmaceutical composition is for use in treating a cancer in an individual. In certain embodiments, the cancer comprises a solid tumor. In certain embodiments, the cancer is selected from the group consisting of lung cancer, colon cancer, glioblastoma, adrenal cancer, uterine cancer, head and neck cancer, pancreatic cancer, and breast cancer. Each possibility represents a separate embodiment of the invention.

According to some embodiments, the cancer is a hematological cancer.

According to some embodiments, the hematological cancer is selected from leukemia including acute myeloid leukemia (AML), chronic myeloid leukemia (CML), acute lymphocytic leukemia (ALL), and chronic lymphocytic leukemia (CLL); lymphoma, including Hodgkin disease, and non-Hodgkin lymphoma; and multiple myeloma.

According to some embodiments, the individual is human.

According to some embodiments of the invention, the use further comprises the use of an agent that downregulates the activity or expression of an immune co-inhibitory receptor.

According to some embodiments, the immune co-inhibitory receptor is selected from the group consisting of PD-1, PD-L1, TIGIT, CTLA-4, LAG3, TIM3, BTLA, VISTA, B7H4, CD96, BY55 (CD 160), LAIR1, SIGLEC10, CD112R, CD112, ILT-4 and 2B4. Each possibility represents a separate embodiment of the invention.

According to some embodiments of the invention, the use further comprises the use in a combination with an anti-endothelial growth factor receptor (EGFR) antibody.

The present invention provides, according to another aspect, a method of increasing surface expression and/or signaling of CD226 in the CD8+ and CD4+ T cells of an individual, the method comprising administering to the individual a therapeutically effective amount of the humanized antibody or antigen binding fragment thereof, or the pharmaceutical composition described herein. In certain embodiments, the CD8+ T cells are tumor infiltrating CD8+ T cells.

The present invention provides, according to another aspect, a method of treating a cancer in an individual in need of such treatment, the method comprising administering to the individual a therapeutically effective amount of the humanized antibody or antigen binding fragment thereof or the pharmaceutical composition. In certain embodiments, the cancer comprises a solid tumor. According to additional embodiments, the cancer is a non-solid tumor.

In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, and lung cancer. In certain embodiments, the method of treating cancer involves preventing or reducing formation, growth or spread of metastases in a subject.

The present invention provides, according to another aspect, a method of treating a cancer in an individual afflicted with a cancer comprising administering to the individual a therapeutically effective amount of the humanized antibody or antigen binding fragment thereof or the pharmaceutical composition, and an inhibitor of PD-1, PD-L1, CTLA-4 or CD112R signaling. In certain embodiments, the cancer comprises a solid tumor. In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, or lung cancer. In certain embodiments, the inhibitor of PD-1 signaling is an antibody or fragment thereof that binds to PD-1. In certain embodiments, the antibody or fragment thereof that binds to PD-1 is Pembrolizumab, Nivolumab, AMP-514, Tislelizumab, Spartalizumab, or a PD-1 binding fragment thereof. In certain embodiments, the inhibitor of PD-1 signaling is an antibody that specifically binds PD-L-I or PD-L-2. In certain embodiments, the antibody that specifically binds PD-L1 or PD-L2 comprises Durvalumab, Atezolizumab, Avelumab, BMS-936559, or FAZ053, or a PD-L1 or PD-L2 binding fragment thereof. In certain embodiments, the inhibitor of PD-1 signaling comprises an Fc-fusion protein that binds PD-1, PD-L1, or PD-L2. In certain embodiments, the Fc-fusion protein comprises AMP-224 or a PD-1 binding fragment thereof. In certain embodiments, the inhibitor of PD-1 signaling comprises a small molecule inhibitor of PD-1, PD-L1, or PD-L2. In certain embodiments, the small molecule inhibitor of PD-1, PD-L1, or PD-L2 signaling comprises on or more of: N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo [b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-ih-indole; L-a-Glutamine, N2,N6-bis(L-seryl-L-asparaginyl-L-threonyl-L-seryl-L-a-glutamyl-L-seryl-L-phenylalanyl)-L-lysyl-L-phenylalanyl-L-arginyl-L-valyl-L-threonyl-L-glutaminyl-L-leucyl-L- alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L-tryptophyl-N-methyl-L- norleucyl-N-methyl-L-norleucyl-L-arginyl-L-cysteinyl-, cyclic (1→14)-thioether; or a derivative or analog thereof.

Also described herein is a method of making composition for treating a cancer in an individual afflicted with cancer comprising admixing the humanized antibody or antigen binding fragment thereof and a pharmaceutically acceptable excipient, carrier, or diluent. In certain embodiments, the cancer comprises a solid tumor. In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, and lung cancer. Also described herein is a method of producing the humanized antibody or antigen binding fragment thereof comprising incubating the cell line described herein in a cell culture medium under conditions sufficient to allow expression and secretion of the humanized antibody or antigen binding fragment thereof.

The present invention further provides, according to an aspect, a method of diagnosing or prognosing cancer in a subject, the method comprises determining the expression level of PVR in a biological sample of said subject using at least one humanized antibody, fragment or scFv as described herein.

The present invention further provides, according to another aspect, a method of determining or quantifying the expression of PVR, the method comprising contacting a biological sample with an antibody or antibody fragment as described herein, and measuring the level of complex formation.

According to some embodiments, the method for detecting or quantifying the expression of PVR comprises the steps of:
i. incubating a sample with an antibody specific to PVR or an antibody fragment thereof comprising at least an antigen-binding portion;
ii. detecting the bound PVR using a detectable probe.

According to some embodiments, the method further comprises the steps of:
iii. comparing the amount of (ii) to a standard curve obtained from a reference sample containing a known amount of PVR; and
iv. calculating the amount of the PVR in the sample from the standard curve.

According to some embodiments, the method comprises indicating a subject as having a PVR positive cancer if the PVR amount is higher than a control or a given reference.

According to some particular embodiments, the sample is a body fluid or solid tissue. In some embodiments, the method is performed in-vitro or ex-vivo.

A kit for measuring the expression of PVR in biological sample is also provided comprising at least one antibody or antibody fragment as described herein and means for measuring PVR expression. In some embodiment, the kit further comprising instruction material directing the use of the kit.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The novel features described herein are set forth with particularity in the appended claims. A better understanding of the characteristics and advantages of the features described herein will be obtained by reference to the following detailed description that sets forth illustrative examples, in which the principles of the features described herein are utilized, and the accompanying drawings of which:

FIGS. 1A-1B. Affinity and competition assays of N56 substitutions. FIG. 1A. Improved affinity for PVR binding by N56E and N56D variants. Biacore assay showing relative and absolute affinity of the N56 substitutions (hIgG4 variants) to PVR-HIS tagged (Sino Cat. no. 10109-H08H). Affinity shift of >25% were considered significant. (*) Relative KDS for each variant were established by dividing the KD of the substitution by the KD of the parental N56 variant (VH0VK0). FIG. 1B compares the potency of chimeric antibodies (5B9 wild type—WT, having IgG4 (S241P) HG) with variable regions carrying the LC CDR2 sequence with single amino acid substitutions (to remove a deamidation site). Potency was measured in a competition assay using parental 5B9. Relative IC50 was determined for each variant by dividing its IC50 by that of the chimeric WT antibody tested in parallel.

FIGS. 2A-2B. Improved cross-reactivity for monkey PVR binding by N56E and N56D variants. To assess maximal binding, saturating dose (10 ug/ml) of the N56 variant monoclonal antibodies (shown at the X axis) were used to stain PVR-expressing cells, and mean fluorescence intensity (MFI) values were determined by FACS analysis. The fold change was calculated by dividing the MFI of each variant by the MFI of the parental antibody (K0). (FIG. 2A) NCI-H1975 (human lung) cells and (FIG. 2B) Vero (African green monkey kidney) cells. Affinity shifts of >25% were considered significant.

FIG. 3. Improved NK activation by N56E and N56T variants. To characterize the N56 substitution variants, a CD107a induction assay was performed using human NK cells from a healthy donor and MDA-MB-231 as target cells at 2:1 ratio. The Abs were added at 600 pM. K0 is the parental clone (N56). All variants led to significant CD107a induction (>240% over isotype IgG1). Additionally, N56 substitutions N56E & N56T significantly improved CD107a induction compared with K0. (*p<0.04, **p<0.01).

FIG. 4. Improved CD8 T cell proliferation by N56E and N56T variants. To characterize the N56 variants, T cell proliferation assay was performed. A549 cancer cells were used at 4:1 effector-to-target ratio, in presence of 2.5 ul/ml PHA-L and using GFSE labeled fresh human PBMCs. All N56 variant monoclonal antibodies (X axis) were added at 4ug/ml and the co-culture was incubated for 96 hrs. Presented are the results of FACS analysis gated 25 on CD8+ T cells. The relative MFI of the GFSE labeling was calculated by dividing the MFI of the of the IgG treated group with that of each variant. As increase in proliferation results in reduced GFSE signal, the Y axis depicts the reciprocal value of this ratio. N56E and N56T variants increased CD8 T cell proliferation significantly over parental clone (K0) marked by #. (*p<0.05, #<0.04, **p<0.01).

FIGS. 5A-5B illustrate the affinities of humanized variants to human PVR as measured by surface plasmon resonance (SPR) (FIG. 5A) or their surface binding to PVR-expressing HEK 293 cells as measured by flow cytometry (FIG. 5B). Shown are absolute results and relative ones, using chimeric N56E mutated parental antibody (N56E VH0/Vk0) as a baseline.

FIGS. 6A-6B show the expression levels (FIG. 6A) and similarity of humanized variants to human variable domain germline sequences (FIG. 6B). The titers of individual variants after transient expression in HEK 293 EBNA cells are shown (FIG. 6A). Variable domain sequence identity of humanized heavy and light chain variants to human germ line sequence is shown (FIG. 6B).

FIGS. 7A-7B compare the biophysical properties of humanized lead variant NB1088 (right column) to the humanized variant NB941 carrying the LC CDR2 5B9 WT sequence (left column). FIG. 7A. and FIG. 7B show reduced generation of acidic species in NB1088 over time following stress test at low pH (FIG. 7A) or at high concentration at 40° C. (FIG. 7B).

FIGS. 8A-8D illustrate the EC₅₀ of NB1088 binding to PVR (FIG. 8A); IC₅₀ of NB1088 inhibition of PVR-TIGIT binding (FIG. 8B); IC₅₀ of NB1088 inhibition of PVR-CD96 binding (FIG. 8C); and IC₅₀ of NB1088 inhibition of PVR-CD226 binding (FIG. 8D).

FIGS. 9A-9B illustrate that NB1088 alone (FIGS. 9A and 9B) and in combination with PD1 inhibition by pembrolizumab (FIG. 9B) increases interferon gamma release in a tumor/T cell coculture system using an antigen specific T cell assay (human papillomavirus; HPV) (FIG. 9A) or a non-specific allogenic T cell assay (FIG. 9B). **p<0.01 one-way Anova.

FIGS. 10A-10B illustrate that NB1088 increases antibody dependent cell cytotoxicity when combined with an endothelial growth factor receptor (EGFR) binding antibody in an EGFR expressing breast cancer cell line A549 (FIG. 10A), and that this coincides with increased interferon gamma release (FIG. 10B). (*p<0.05, **p<0.01 one-way Anova.

FIGS. 11A-11B illustrate that PVR expressing tumor cell lines (A549 or CaSki) can induce downregulation of CD226 expressed on CD8 T-cells (FIG. 11A) and NK-cells (FIG. 11B) that can be restored by NB1088 whereas anti-TIGIT cannot (FIGS. 11A and 11B). FIG. 11A shows results obtained in coculture system using an antigen specific T cell assay (human papillomavirus; HPV), or a non-specific allogenic T cell assay.

FIGS. 12A-12B illustrate NB1088 dependent increase in interferon gamma release by allo- or antigen-responsive CD8+ T cells (FIG. 12A) or that NB1088 induction of Antibody-dependent cellular cytotoxicity(ADCC)-responsive NK cells in the presence of EGFR blocking (FIG. 12B) depend on CD226 activity, and that in both cases NB1088 shows superior activity over TIGIT inhibition (both Abs are at 10 ug/ml).

FIGS. 13A-13E illustrate that NB1088 has efficacy as monotherapy in a humanized mouse model of pancreatic cancer (FIG. 13A) that rivals pembrolizumab (anti-PD-1, given at the standard dose established for these models) (FIG. 13B) and in a humanized mouse model of lung adenocarcinoma (FIG. 13C, humanized mice), and that in this lung adenocarcinoma model efficacy depends on the presence of human immune cells (FIG. 13D, non-humanized mice) and correlates with increased CD226 expression on tumor infiltrating CD8+ T cells (FIG. 13E). (*p<0.05, ***p<0.001 by two-way Anova).

FIGS. 14A-14D relate to the model at FIGS. 13C and 13E and illustrate that NB1088 increases the frequency of interferon gamma positive (FIG. 14A) and interferon gamma/CD107a double positive (FIG. 14B) CD8 TIL cells and that NB1088 increases the frequencies of interferon gamma+/CD226+ double positive CD8 T-cells (FIG. 14D), but not interferon gamma+/CD226− single positive CD8 T-cells (FIG. 14C). *p=0.0210 ***<0.0001 unpaired TTEST.

FIGS. 15A-15B illustrate the pharmacokinetics of NB1088 (FIG. 15A) and corresponding pharmacodynamic changes in CD226 surface expression (FIG. 15B) on circulating CD4 T cells in cynomolgus monkey that were treated with various doses of the antibody either once (for the 20 and 50 mg/kg doses), or 4 times, one week apart (for the 200 mg/kg dose).

FIG. 16 shows the expression of human PVR across biopsies of different cancer types measured by immunohistochemistry and evaluated by H-score.

FIG. 17 is a general schematic drawing of CAR-T constructs. The scFv includes the heavy and light chains (VH and VL, respectively) of the humanized antibodies according to the invention.

FIG. 18 Illustrates robust Interleukin 2 (IL2) secretion of Jurkat cells overexpressing uPVR CAR-T constructs over parental Jurkat cells. Parental Jurkat cells or Jurkat cells overexpressing uPVR CAR-T constructs H4K2-NTX1088C or H3K4-NTX1034C (40K cells/well) were incubated with A549 or MDA-231 cells at 1:1 E:T ratio for 24 hours IL2 secretion was quantified using Biolegend hIL-2 (cat 431804). Both CAR-T drivers increased the IL2 secretion over 100-fold over the parental Jurkat cells in presence of the indicated targets.

FIGS. 19A-19C Illustrate increased target cell killing by anti PVR (uPVR) CAR-T. A549 (FIG. 19A) or MDA-231 (FIG. 19B) cells (both at 200K cells) were plated in a 12 well plate with CAR-T-PVR variants NTX-1088C and NTX-1034C in NK media for 72 hours, at E:T ratios of 0.4 and 0.8 to 1, respectively (based on GFP positivity). Tumor cell killing was assessed using the standard CTG protocol (Promega G9241).

FIG. 20. Illustrates efficient hematological target cell killing by uPVR CAR-Ts. K562 (an AML model) cells were incubated with uPVR CAR-T variants NTX-1088C and NTX-1034C in RPMI+IL-2 for 18 hours at E:T ratios indicated at the X axis. Tumor cell killing was assessed using the flow cytometry. Significant target cell killing was observed for both CAR-T drivers.

### DETAILED DESCRIPTION OF THE INVENTION

The present invention provides humanized monoclonal antibodies which recognize poliovirus receptor (PVR). Advantageously, the antibodies of the invention are almost fully humanized, thus avoiding the risk of adverse immune response towards the antibodies and are therefore safe for in-vivo use in humans. The antibodies of the invention are characterized by having unique CDR sequence and novel humanized framework sequences and design. More specifically, the monoclonal antibodies provided by the present invention have specific combinations of CDRs and non-fully-humanized framework sequences, and possess unique properties and improved safety and potency over known anti-PVR antibodies.

Some of the variants described herein possess increased producibility and expresses at higher levels compared to other humanized PVR antibodies comprising the same CDR regions. Also disclosed herein are methods of using these antibodies to treat a cancer in an individual.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The term “PVR” as used herein refers to the poliovirus receptor, also known as CD155 (cluster of differentiation 155), Protein ID: Q92692 according to some embodiments. The PVR is a transmembrane glycoprotein with a N-terminal signal sequence, three extracellular immunoglobulin (Ig)-like domains, a transmembrane domain and a cytoplasmic tail. It has a molecular size of approximately 80 kDa and a structure composed of three Ig-like domains, specifically an outermost V-like domain followed by two C2-like domains. The humanized antibodies described herein have affinity to human PVR (hPVR). In some embodiments, the antibodies have some affinity to PVR proteins from other animals, in particular primates. Advantageously, the affinity for other primates, such as the African green monkey, enables further testing of the humanized antibodies for safety and efficiency in non-clinical trials. No affinity was seen for PVR from more evolutionarily distant animals, such as rodents.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. According to some embodiments the individual is a mammal. According to some embodiments, the mammal is a mouse, rat, rabbit, dog, cat, horse, cow, sheep, pig, goat, llama, alpaca, or yak. According to some embodiments, the individual is a human.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein the term an “effective amount” refers to the amount of a therapeutic that causes a biological effect when administered to a mammal. Biological effects include, but are not limited to, inhibition or blockade of a receptor ligand interaction (e.g., PVR-TIGIT, PD-1-PD-L1/PD-L-2), inhibition of a signaling pathway, reduced tumor growth, reduced tumor metastasis, or prolonged survival of an animal bearing a tumor. A “therapeutic amount” is the concertation of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

As used herein “checkpoint inhibitor” refers a drug that inhibits a biological molecule (“checkpoint molecule”) produced by an organism that negatively regulates the anti-tumor/cancer activity of T cells in the organism. Checkpoint molecules include without limitation PD-1, PD-L-1, PD-L-2, CTLA4, TIM-3, LAG-3, VISTA, SIGLEC7, PVR, TIGIT, IDO, KIR, A2AR, B7-H3, B7H4, CEACAM1, NOX2, CD112R, and CD112.

Among the provided antibodies are monoclonal antibodies, polyclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 June 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

The term “antigen” as used herein refers to a molecule or a portion of a molecule capable of eliciting antibody formation and being specifically bound by an antibody. An antigen may have one or more than one epitope. The specific binding referred to above is meant to indicate that the antigen will react, in a highly selective manner, with its corresponding antibody and not with the multitude of other antibodies which may be evoked by other antigens. An antigen according to some embodiments of the present invention is a human PVR.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. According to some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. According to some embodiments, some FR residues in a humanized antibody are substituted with corresponding residues from a non-human antibody (e.g., the antibody from which the CDR residues are derived), e.g., to restore or improve antibody specificity or affinity.

A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. According to some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “homologous”, “homology” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved antibody-dependent cellular cytotoxicity (ADCC) or complement dependent cytotoxicity (CDC).

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10⁻⁸ M or less, e.g., from 10⁻⁸ M to 10⁻¹³ M, e.g., from 10⁻⁹ M to 10⁻¹³ M) for the antibody target, human poliovirus receptor (CD155). K_{D} can be measured by any suitable assay. In certain embodiments, KD can be measured using surface plasmon resonance (SPR) assays (e.g., using a BIACORE®-2000 or a BIACORE®-3000).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. According to some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; Al 18 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol. In certain embodiments, the master cell bank comprises: (a) a CHO cell line comprising a nucleic acid encoding an antibody with (i) a heavy chain amino acid sequence set forth by any one of SEQ ID NOs: 1, 3, 4, 5 or 6; and (ii) a light chain amino acid sequence set forth by any one of SEQ ID NOs: 2, 7, 8 or 9 integrated at a genomic location; and (b) a cryoprotectant. In certain embodiments, the cryoprotectant comprises glycerol. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

### Antibodies Described Herein

In a certain aspect described herein is an anti-human PVR (anti-hPVR) antibody or antigen binding fragment thereof. In certain embodiments, the antibody or antigen binding fragment thereof binds to the human PVR at the PVR-TIGIT interface. In certain embodiments, the anti-hPVR antibody or antigen binding fragment thereof is able to compete with any one or more of TIGIT, CD96, and CD226.

In certain embodiments, the EC₅₀ of a humanized antibody or antigen binding fragment thereof for binding human PVR is less than about 10 nM, 9 nM, 8 nM, 7 nM, 6 nM, 5 nM, 4 nM, 3 nM, 2 nM, 1 nM, 0.5 nM, 0.1 nM, 0.05 nM, or 0.01 nM. In certain embodiments, the EC₅₀ of the hPVR antibody for binding to PVR is between about 5 nM and 1 nM, between about 5 nM and about 2 nM, between about 4 nM and about 2 nM, between about 4 nM and about 3 nM, or between about 3 nM and about 2 nM.

Half maximal effective concentration (EC₅₀) refers to the concentration of the antibody which induces a response halfway between the baseline and maximum after a specified exposure time.

According to some embodiments, the antibody is a recombinant antibody. According to specific embodiments, the antibody is a recombinant humanized monoclonal antibody.

According to some embodiments, the humanized antibody or antigen binding fragment thereof comprises a heavy chain sequence selected from the group consisting of SEQ ID NO: 1, SEQ ID NO: 3, SEQ ID NO: 4, SEQ ID NO: 5, and SEQ ID NO: 6.

According to some embodiments, the humanized antibody or antigen binding fragment thereof comprises a light chain sequence selected from the group consisting of SEQ ID NO: 2, SEQ ID NO: 7, SEQ ID NO: 8, and SEQ ID NO: 9.

According to some embodiments, the humanized antibody or antigen binding fragment thereof is NB1088 (SEQ ID NO: 1 and SEQ ID NO: 2).

In one aspect described herein is a humanized antibody or antigen binding fragment thereof, wherein the antibody or antigen binding fragment thereof comprises a heavy chain and a light chain, wherein the heavy chain comprises an amino acid sequence at least about 90% identical to a sequence selected from the group consisting of SEQ ID NO: 1, SEQ ID NO: 3, SEQ ID NO: 4, SEQ ID NO: 5 and SEQ ID NO: 6; and wherein the light chain comprises an amino acid sequence at least about 90% identical to a sequence selected from the group consisting of SEQ ID NO: 2, SEQ ID NO: 7, SEQ ID NO: 8 and SEQ ID NO: 9, wherein the antibody or antigen binding fragment thereof binds to human poliovirus receptor (CD155).

In another aspect described herein is a humanized antibody or antigen binding fragment thereof, wherein the antibody or antigen binding fragment thereof comprises a heavy chain and an immunoglobulin light chain, wherein the heavy chain comprises an amino acid sequence at least about 90% identical to that set forth in SEQ ID NO: 1, and wherein the light chain comprises an amino acid sequence at least about 90% identical to that set forth in SEQ ID NO: 2, wherein the antibody or antigen binding fragment thereof binds to human poliovirus receptor (CD155).

In certain embodiments, described herein is a humanized antibody or antigen binding fragment thereof, wherein the antibody or antigen binding fragment thereof comprises a heavy chain and a light chain, wherein the heavy chain comprises an amino acid sequence at least about 95% identical to a sequence selected from the group consisting of SEQ ID NO: 1, SEQ ID NO: 3, SEQ ID NO: 4, SEQ ID NO: 5 and SEQ ID NO: 6; and wherein the light chain comprises an amino acid sequence at least about 95% identical to a sequence selected from the group consisting of SEQ ID NO: 2, SEQ ID NO: 7, SEQ ID NO: 8 and SEQ ID NO: 9, wherein the antibody or antigen binding fragment thereof binds to human poliovirus receptor (CD155).

In certain embodiments, described herein is a humanized antibody or antigen binding fragment thereof, wherein the antibody or antigen binding fragment thereof comprises a heavy chain and a light chain, wherein the heavy chain comprises an amino acid sequence at least about 95%, 96%, 97%, 98%, or 99% identical to a sequence selected from the group consisting of SEQ ID NO: 1, SEQ ID NO: 3, SEQ ID NO: 4, SEQ ID NO: 5 and SEQ ID NO: 6, and wherein the light chain comprises an amino acid sequence at least about 95%, 96%, 97%, 98%, or 99% identical to a sequence selected from the group consisting of SEQ ID NO: 2, SEQ ID NO: 7, SEQ ID NO: 8 and SEQ ID NO: 9, wherein the antibody or antigen binding fragment thereof binds to human poliovirus receptor (CD155). Each possibility represents a separate embodiment of the invention.

In certain embodiments, described herein is a humanized antibody or antigen binding fragment thereof, wherein the antibody or antigen binding fragment thereof comprises a heavy chain and a light chain, wherein the heavy chain comprises an amino acid sequence at least about 98% identical to that set forth in SEQ ID NO: 1, and wherein the light chain comprises an amino acid sequence at least about 98% identical to that set forth in SEQ ID NO:2, wherein the antibody or antigen binding fragment thereof binds to human poliovirus receptor (CD155).

In certain embodiments, described herein is a humanized antibody or antigen binding fragment thereof, wherein the antibody or antigen binding fragment thereof comprises a heavy chain and a light chain, wherein the heavy chain comprises an amino acid sequence at least about 99% identical to that set forth in SEQ ID NO: 1, and wherein the light chain comprises an amino acid sequence at least about 99% identical to that set forth in SEQ ID NO:2, wherein the antibody or antigen binding fragment thereof binds to human poliovirus receptor (CD155).

In certain embodiments, described herein is a humanized antibody or antigen binding fragment thereof, wherein the antibody or antigen binding fragment thereof comprises a heavy chain and a light chain, wherein the heavy chain comprises an amino acid sequence identical to that set forth in SEQ ID NO: 1, and wherein the light chain comprises an amino acid sequence identical to that set forth in SEQ ID NO:2, wherein the antibody or antigen binding fragment thereof binds to human poliovirus receptor(CD155).

According to some embodiments, the antibody or antigen binding fragment thereof comprises the heavy chain sequence set forth in SEQ ID NO: 1, and the light chain sequence set forth in SEQ ID NO: 7. According to some embodiments, the antibody or antigen binding fragment thereof comprises the heavy chain sequence set forth in SEQ ID NO: 1, and the light chain sequence set forth in SEQ ID NO: 8. According to some embodiments, the antibody or antigen binding fragment thereof comprises the heavy chain sequence set forth in SEQ ID NO: 1, and the light chain sequence set forth in SEQ ID NO: 9. According to some embodiments, the antibody or antigen binding fragment thereof comprises the heavy chain sequence set forth in SEQ ID NO: 3, and the light chain sequence set forth in SEQ ID NO: 2. According to some embodiments, the antibody or antigen binding fragment thereof comprises the heavy chain sequence set forth in SEQ ID NO: 3, and the light chain sequence set forth in SEQ ID NO: 7. According to some embodiments, the antibody or antigen binding fragment thereof comprises the heavy chain sequence set forth in SEQ ID NO: 3, and the light chain sequence set forth in SEQ ID NO: 8. According to some embodiments, the antibody or antigen binding fragment thereof comprises the heavy chain sequence set forth in SEQ ID NO: 3, and the light chain sequence set forth in SEQ ID NO: 9. According to some embodiments, the antibody or antigen binding fragment thereof comprises the heavy chain sequence set forth in SEQ ID NO: 4, and the light chain sequence set forth in SEQ ID NO: 2. According to some embodiments, the antibody or antigen binding fragment thereof comprises the heavy chain sequence set forth in SEQ ID NO: 4, and the light chain sequence set forth in SEQ ID NO: 7. According to some embodiments, the antibody or antigen binding fragment thereof comprises the heavy chain sequence set forth in SEQ ID NO: 4, and the light chain sequence set forth in SEQ ID NO: 8. According to some embodiments, the antibody or antigen binding fragment thereof comprises the heavy chain sequence set forth in SEQ ID NO: 4, and the light chain sequence set forth in SEQ ID NO: 9. According to some embodiments, the antibody or antigen binding fragment thereof comprises the heavy chain sequence set forth in SEQ ID NO: 5, and the light chain sequence set forth in SEQ ID NO: 2. According to some embodiments, the antibody or antigen binding fragment thereof comprises the heavy chain sequence set forth in SEQ ID NO: 5, and the light chain sequence set forth in SEQ ID NO: 7. According to some embodiments, the antibody or antigen binding fragment thereof comprises the heavy chain sequence set forth in SEQ ID NO: 5, and the light chain sequence set forth in SEQ ID NO: 8. According to some embodiments, the antibody or antigen binding fragment thereof comprises the heavy chain sequence set forth in SEQ ID NO: 5, and the light chain sequence set forth in SEQ ID NO: 9. According to some embodiments, the antibody or antigen binding fragment thereof comprises the heavy chain sequence set forth in SEQ ID NO: 6, and the light chain sequence set forth in SEQ ID NO: 2. According to some embodiments, the antibody or antigen binding fragment thereof comprises the heavy chain sequence set forth in SEQ ID NO: 6, and the light chain sequence set forth in SEQ ID NO: 7. According to some embodiments, the antibody or antigen binding fragment thereof comprises the heavy chain sequence set forth in SEQ ID NO: 6, and the light chain sequence set forth in SEQ ID NO: 8. According to some embodiments, the antibody or antigen binding fragment thereof comprises the heavy chain sequence set forth in SEQ ID NO: 6, and the light chain sequence set forth in SEQ ID NO: 9.

According to some embodiments, the humanized antibody or antigen binding fragment thereof comprising a heavy chain comprises the amino acid sequence 10 QVQLVQSGAE(L/V)KKPGASVK(I/V)SCKATGYTFSNYWIEW(I/V)(K/R)QAPGQGLE W(I/M)GEIFPGSGRINFNEKFKGR(A/V)TFTADTSI(D/S)T(T/A)YM(Q/E)LS(S/R)L(T/R) SDD(S/T)AVYYCARTKIYGNSFDYWGQGT(T/L)VTVSS (SEQ ID NO: 47); and a light chain comprises the amino acid sequence DI(M/Q)MTQSPS(F/S)LSASVGDRVTITC(K/R)ASQDVGTAV(V/A)WYQQKPGKAPK(L/S)LIYWASSRHEGVP(D/S)RF(T/S)GSGSGTDFTLTISSLQ(S/P)EDFA(D/T)YFCQQYS RYPLTFGQGT KLEIK (SEQ ID NO: 48).

According to some embodiments, the heavy chain comprises an amino acid sequence set forth in SEQ ID NO: 47, wherein position 11 is L, or position 20 is I, or position 37 is I, or position 38 is K, or position 48 is I, or position 68 is A, or position 77 is D, or position 79 is T, or position 82 is Q, or position 85 is S, or position 87 is T, or position 91 is S, or position 114 is T, or any combination thereof. Each possibility represents a separate embodiment of the invention.

According to some embodiments, the heavy chain comprises an amino acid sequence set forth in SEQ ID NO: 47, wherein position 11 is V, or position 20 is I, or position 37 is I, or position 38 is K, or position 48 is I, or position 68 is A, or position 77 is D, or position 79 is T, or position 82 is E, or position 85 is R, or position 87 is R, or position 91 is T, or position 114 is L, or any combination thereof. Each possibility represents a separate embodiment of the invention.

According to some embodiments, the heavy chain comprises an amino acid sequence set forth in SEQ ID NO: 47, wherein position 11 is V, or position 20 is V, or position 37 is V, or position 38 is R, or position 48 is M, or position 68 is V, or position 77 is S, or position 79 is A, or position 82 is E, or position 85 is R, or position 87 is R, or position 91 is T, or position 114 is L, or any combination thereof. Each possibility represents a separate embodiment of the invention.

According to some embodiments, the heavy chain comprises an amino acid sequence set forth in SEQ ID NO: 47, wherein position 11 is V, or position 20 is V, or position 37 is I, or position 38 is K, or position 48 is I, or position 68 is V, or position 77 is S, or position 79 is T, or position 82 is E, or position 85 is R, or position 87 is R, or position 91 is T, or position 114 is L, or any combination thereof. Each possibility represents a separate embodiment of the invention.

According to some embodiments, the heavy chain comprises an amino acid sequence set forth in SEQ ID NO: 47, wherein position 11 is V, or position 20 is V, or position 37 is V, or position 38 is R, or position 48 is I, or position 68 is V, or position 77 is S, or position 79 is T, or position 82 is E, or position 85 is R, or position 87 is R, or position 91 is T, or position 114 is L, or any combination thereof. Each possibility represents a separate embodiment of the invention.

According to some embodiments, the light chain comprises an amino acid sequence set forth in SEQ ID NO: 48, wherein position 3 is M, or position 10 is F, or position 24 is K, or position 34 is V, or position 46 is L, or position 60 is D, or position 63 is T, or position 80 is S, or position 85 is D, or any combination thereof. Each possibility represents a separate embodiment of the invention.

According to some embodiments, the light chain comprises an amino acid sequence set forth in SEQ ID NO: 48, wherein position 3 is Q, or position 10 is S, or position 24 is K, or position 34 is V, or position 46 is L, or position 60 is D, or position 63 is S, or position 80 is P, or position 85 is D, or any combination thereof. Each possibility represents a separate embodiment of the invention.

According to some embodiments, the light chain comprises an amino acid sequence set forth in SEQ ID NO: 48, wherein position 3 is Q, or position 10 is S, or position 24 is R, or position 34 is V, or position 46 is L, or position 60 is S, or position 63 is S, or position 80 is P, or position 85 is T, or any combination thereof. Each possibility represents a separate embodiment of the invention.

According to some embodiments, the light chain comprises an amino acid sequence set forth in SEQ ID NO: 48, wherein position 3 is Q, or position 10 is S, or position 24 is R, or position 34 is A, or position 46 is L, or position 60 is S, or position 63 is S, or position 80 is P, or position 85 is T, or any combination thereof. Each possibility represents a separate embodiment of the invention.

According to some embodiments, the humanized antibody or antigen binding fragment thereof comprising a heavy chain variable region and a light chain variable region, the heavy chain comprises an amino acid sequence set forth in SEQ ID NO: 47, wherein position 11 is V, or position 20 is V, or position 37 is V, or position 38 is R, or position 48 is I, or position 68 is V, or position 77 is S, or position 79 is T, or position 82 is E, or position 85 is R, or position 87 is R, or position 91 is T, or position 114 is L, or any combination thereof; and the light chain comprises an amino acid sequence set forth in SEQ ID NO: 48, wherein position 3 is Q, or position 10 is S, or position 24 is K, or position 34 is V, or position 46 is L, or position 60 is D, or position 63 is S, or position 80 is P, or position 85 is D, or any combination thereof.

According to additional embodiments, the heavy chain CDR1 sequence is GYTFSNYWIE (SEQ ID NO: 58).

According to some embodiments the human constant regions of the antibody are selected from the group consisting of: human IgG1, human IgG2, human IgG3, and human IgG4.

According to some embodiments the human constant regions of the antibody are selected from the group consisting of: human IgG1 and human IgG4.

According to some embodiments, the humanized antibody is an IgG4 antibody comprising a heavy chain sequences set forth in SEQ ID NO: 49, or a sequence having at least 90% identity. According to some embodiments, the humanized antibody is an IgG1 comprising a heavy chain sequence set forth in SEQ ID NO: 50 or a sequence having at least 90% identity.

According to some embodiments, the humanized antibody comprising a light chain sequence set forth in SEQ ID NO: 49.

### Therapeutic Methods

In certain embodiments, disclosed herein, are anti-hPVR antibodies useful for the treatment of a cancer or tumor. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In certain embodiments, the anti-hPVR antibodies and antigen binding fragments described herein are for use in the manufacture of a medicament for or for use in a method of treating a PVR positive cancer.

In certain embodiments, the anti-hPVR antibody or antigen binding fragments described herein is for treating a cancer or tumor that is refractory to treatment with a checkpoint inhibitor as a monotherapy. Refractory cancer refers to a cancer/tumor that develops progressive disease despite treatment with the checkpoint inhibitor alone. In certain embodiments the checkpoint inhibitor is a PD-1, PD-L1, or PD-L2 inhibitor. In certain embodiments, the PD-1, PD-L1, or PD-L2 inhibitor is an antibody or antigen binding fragment that specifically binds PD-1 (CD279) comprises Pembrolizumab, Nivolumab, AMP-514, Spartalizumab, Tislelizumab (BGB-A317), or a PD-1 (CD279) binding fragment thereof. In certain embodiments PD-1, PD-L1, or PD-L2 inhibitor is a PD-L2 Fc fusion protein (e.g., AMP-224). In certain embodiments PD-1, PD-L1, or PD-L2 inhibitor comprises an antibody or PD-L1 binding fragment that specifically binds PD-L1 (CD274). In certain embodiments, the antibody or antigen binding fragment that specifically binds to PD-L1 (CD274) comprises Durvalumab (MEDI 4376), Atezolizumab, Avelumab, BMS-936559, or FAZ053, or a PD-L1 (CD274) binding fragment thereof. In certain embodiments PD-1, PD-L1, or PD-L2 inhibitor comprises an antibody or PD-L2 binding fragment thereof that specifically binds PD-L2 (CD273). In certain embodiments PD-1, PD-L1, or PD-L2 inhibitor comprises one or more small molecule inhibitor such as N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo [b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-a-glutamyl-L-seryl-L-phenylalanyl)-L-lysyl-L-phenylalanyl-L-arginyl-L-valyl-L-threonyl-L-glutaminyl-L- leucyl-L-alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L-tryptophyl-N-methyl-L- norleucyl-N-methyl-L-norleucyl-L-arginyl-L-cysteinyl-, cyclic (1→14)-thioether; or a derivative or analog thereof.

In certain embodiments, the anti-hPVR antibody or antigen binding fragments thereof are for use in combination with a PD-1, PD-L1, or PD-L2 inhibitor. In certain embodiments, the PD-1, PD-L1, or PD-L2 inhibitor is an antibody or antigen binding fragment that specifically binds PD-1 (CD279) comprises Pembrolizumab, Nivolumab, AMP-514, Spartalizumab, Tislelizumab (BGB-A317), or a PD-1 (CD279) binding fragment thereof. In certain embodiments PD-1, PD-L1, or PD-L2 inhibitor is a PD-L2 Fc fusion protein (e.g., AMP-224). In certain embodiments PD-1, PD-L1, or PD-L2 inhibitor comprises an antibody or PD-L-I binding fragment that specifically binds PD-L-1 (CD274). In certain embodiments, the antibody or antigen binding fragment that specifically binds to PD-L-i (CD274) comprises Durvalumab (MEDI 4376), Atezolizumab, Avelumab, BMS-936559, or FAZ053, or a PD-L-1 (CD274) binding fragment thereof. In certain embodiments PD-1, PD-L1, or PD-L2 inhibitor comprises an antibody or PD-L2 binding fragment thereof that specifically binds PD-L2 (CD273). In certain embodiments PD-1, PD-L1, or PD-L2 inhibitor comprises one or more small molecule inhibitor such as N-{2-[({2-methoxy-6-[(2-methyl[1,1′-biphenyl]-3-yl)methoxy]pyridin-3-yl}methyl)amino]ethyl}acetamide (BMS 202); (2-((3-cyanobenzyl)oxy)-4-((3-(2,3-dihydrobenzo [b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-a-glutamyl-L-seryl-L-phenylalanyl)-L-lysyl-L-phenylalanyl-L-arginyl-L-valyl-L-threonyl-L-glutaminyl-L- leucyl-L-alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl)methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L-tryptophyl-N-methyl-L- norleucyl-N-methyl-L-norleucyl-L-arginyl-L-cysteinyl-, cyclic (1→14)-thioether; or a derivative or analog thereof.

In certain embodiments, the anti-hPVR antibody or antigen binding fragments thereof are for use in combination with an EGFR inhibitor or an EGFR binding antibody.

In certain embodiments, the anti-hPVR antibodies or antigen binding fragments thereof are for use in treating a cancer or tumor. In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and/or liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, or lung cancer.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered, its persistence in the blood circulation, and the judgment of the treating physician.

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated.

The antibodies of the present invention can be used in CAR-based adoptive immunotherapies that utilizes engineered lymphocytes comprising the CAR for treating cancer. CAR-T system is described herein as a non-limiting example.

The T cell therapy utilizes a chimeric antigen receptor (CAR) in the treatment of cancer or tumors (i.e., CAR-T cell therapy). CAR-T cell therapy is a cellular immunotherapy which involves administration to a cancer patient genetically engineered T-cells that act on tumor cells and cause apoptosis of the tumor cells. The genetically engineered T cells are prepared by expressing on a T cell a CAR having variable regions of an antibody (VL and VH) combined with an intracellular domain, such as fragment of a CD3ζ chain sequence, using gene transfer technique. CAR is a general term for a chimeric protein in which a light chain and a heavy chain of a variable region of a monoclonal antibody specific for a tumor antigen are linked to each other, which are then linked to a T-cell receptor (TCR) chain at the C-terminal side.

According to some embodiments, the CAR comprises at least one protein domain selected from the group consisting of a CD8 Stalk domain, a CD28 TM domain, a 41BB domain, and a CD3(domain. According to some embodiments, the CAR comprises a CD8 Stalk domain. According to some embodiments, the CAR comprises a CD28 TM domain. According to some embodiments, the CAR comprises a CD3ζ signaling domain. According to some embodiments, the CAR comprises a 41BB domain. According to specific embodiments, the CAR comprises a CD8 Stalk domain, a CD28 TM domain, a 41BB domain, and a CD3ζ domain.

According to some embodiments, a chimeric antigen receptor (CAR) comprising the heavy chain variable region (VH) and the light chain variable region (VL) according to the invention is provided. According to certain embodiments, a genetically modified lymphocyte having the CAR being expressed on its surface is provided. According to some specific embodiments, a genetically modified T cell having the CAR being expressed on its surface (CAR-T cell) is provided.

According to some embodiments, the CAR comprises a combination of heavy and light chain variable regions, the heavy chain variable region comprises an amino acid sequence with at least 90%, 92%, 94%, 96%, or 98% sequence identity to a sequence selected from the group consisting of SEQ ID NO: 1, SEQ ID NO: 3, SEQ ID NO: 4, SEQ ID NO: 5 and SEQ ID NO: 6; and the light chain variable region comprises an amino acid sequence with at least 90%, 92%, 94%, 96%, or 98% sequence identity to a sequence selected from the group consisting of SEQ ID NO: 2, SEQ ID NO: 7, SEQ ID NO: 8 and SEQ ID NO: 9.

According to some embodiments, the CAR comprises a heavy chain variable region comprising an amino acid sequence selected from the group consisting of SEQ ID NO: 1, SEQ ID NO: 3, SEQ ID NO: 4, SEQ ID NO: 5 and SEQ ID NO: 6, and a light chain variable region comprising an amino acid sequence selected from the group consisting of SEQ ID NO: 2, SEQ ID NO: 7, SEQ ID NO: 8 and SEQ ID NO: 9.

According to some embodiments, the CAR comprises a combination of a humanized antibody heavy and light chain variable regions, wherein the combination is selected from the group consisting of:
i. a heavy chain variable region sequence set forth in SEQ ID NO: 1 and a light chain variable region sequence set forth in SEQ ID NO: 9;
ii. a heavy chain variable region sequence set forth in SEQ ID NO: 3 and a light chain variable region sequence set forth in SEQ ID NO: 9;
iii. a heavy chain variable region sequence set forth in SEQ ID NO: 4 and a light chain variable region sequence set forth in SEQ ID NO: 9;
iv. a heavy chain variable region sequence set forth in SEQ ID NO: 5 and a light chain variable region sequence set forth in SEQ ID NO: 9; and
v. a heavy chain variable region sequence set forth in SEQ ID NO: 6 and a light chain variable region sequence set forth in SEQ ID NO: 9.

According to some embodiments, the CAR comprises a heavy chain variable region sequence selected from the group consisting of SEQ ID NO: 1, 3, 4, 5, and 6, and a light chain variable region sequence set forth in SEQ ID NO: 9, a transmembrane domain, and an intracellular T cell signaling domain.

According to some embodiments, the CAR comprises a scFv sequence set forth in SEQ ID NO: 56 or SEQ ID NO: 57, or an analog thereof having at least 90%, 92%, 94%, 96%, or 98% sequence similarity to any of said sequences is provided. According to a certain aspect, the present invention provides a cell comprising the CAR described herein. According to some embodiments, the cell expresses or capable of expressing the CAR of the present invention. According to some embodiments, the cell is a lymphocyte. According to some embodiments, the cell is selected from a T cell and a natural killer (NK) cell.

According to some embodiments, a lymphocyte engineered to express the CAR described herein is provided. According to some embodiments, a T cell engineered to express the CAR described herein is provided.

According to additional embodiments, an NK cell engineered to express the CAR described herein is provided.

The present invention further discloses methods for diagnosing and prognosing cancer.

According to an aspect, the present invention provides a diagnostic and/or prognostic method of cancer or infectious disease in a subject, the method comprises the step of determining the expression level of PVR in a biological sample of said subject using at least one antibody as described herein.

### Pharmaceutically Acceptable Excipients, Carriers, and Diluents

In certain embodiments the anti-PVR antibodies of the current disclosure are included in a pharmaceutical composition comprising one or more pharmaceutically acceptable excipients, carriers, and diluents. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

Typically, the antibodies and fragments and conjugates thereof of the present invention comprising the antigen binding portion of an antibody or comprising another polypeptide including a peptide-mimetic will be suspended in a sterile saline solution for therapeutic uses. The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles.

In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In certain embodiments, the lymphocytes bearing the CAR described herein are shipped/stored before use. The cells are usually cryopreserved when not used immediately. Cryopreservation methods and storage media suitable for cells bearing CAR are known in the art, see for example, Wang, et al. 2019 May; 21(5):566-578.

Also described herein are kits comprising one or more of the antibodies described herein in a suitable container and one or more additional components selected from: instructions for use; a diluent, an excipient, a carrier, and a device for administration. In some embodiments, the kit comprises means for measuring expression of human PVR.

In certain embodiments, described herein is a method of preparing a cancer treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### EXAMPLES

The following illustrative examples are representative of embodiments of compositions and methods described herein and are not meant to be limiting in any way.

### Example 1—Improved Affinity of PVR Binding by N56E and N56D Variants

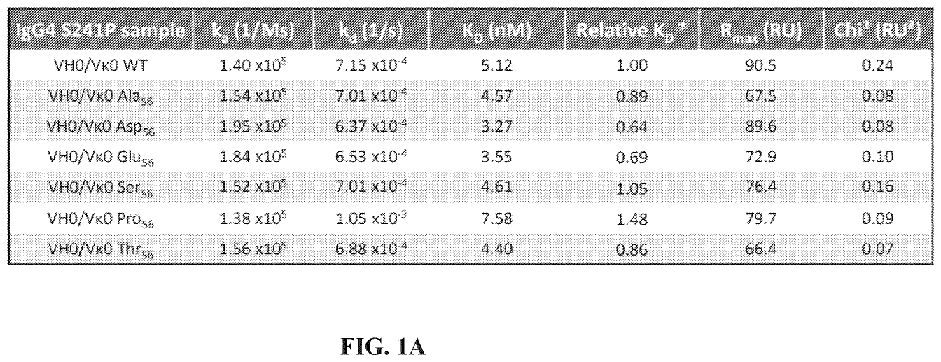

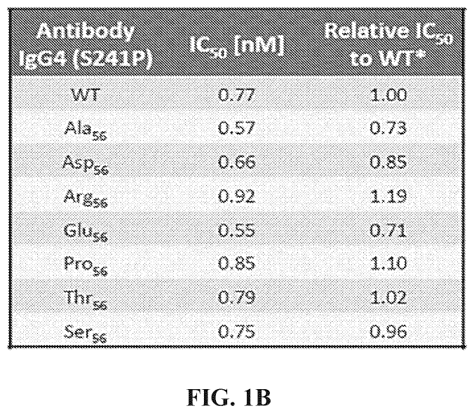

The variable region of the chimeric anti-PVR antibody 5B9, disclosed in WO2017149538, carries a deamidation sequence (Asparagine-Glycine), in CDR2 of the light chain (WASSRHNG, SEQ ID NO: 17). Seven chimeric variants were generated by introducing a point mutation at residue asparagine N56. To assess the binding affinity of the N56 substitution variants, wild type (WT) and substitution variants IgG4 (S241P) monoclonal antibodies were immobilized on Protein A capture chip. Binding was tested for the analyte PVR conjugated to Histidine tag (PVR-HIS, Sino Cat. no. 10109-H08H). Dilution range: Five point two-fold dilution from 50 nM to 3.125 nM. Conditions used: Instrument: Biacore T200 (serial no. 1909913) running Biacore T200 Evaluation Software V2.0.1. Running buffer: HBS-P+, 300 mM NaCl, 1 mg/ml BSA. Flow rate: 30 pl/min. Association: 350 s, Dissociation: 800 s. Regeneration: 10 mM glycine pH 1.5. Analysis: 1:1 binding. Relative K_{D} for each substitution was established by dividing the K_{D} of the substitution to the K_{D} of the parental N56 variant (VH0VK0). Significant (>25%) improvement of affinity was noted for the N56E (Asp) and N56D (Glu) variants (FIG. 1A). The binding of chimeric variants to human PVR expressed on HEK 293 EBNA cells was assessed by flow cytometry in a competition assay using parental 5B9 antibody (WT, FIG. 1B).

### Example 2—Improved Cross-Reactivity for Monkey PVR Binding by N56E and N56D Variants

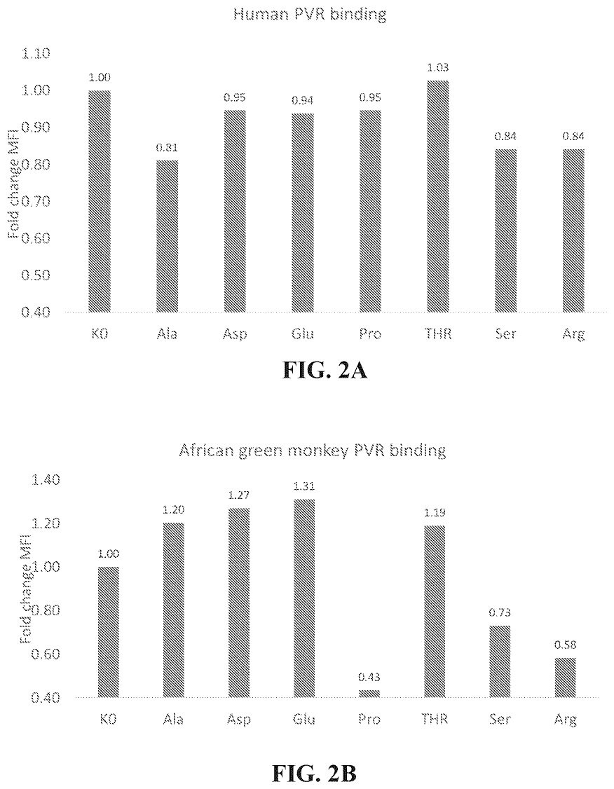

The binding of the N56 substitution variant antibodies to cell bound PVR of human (protein id: Q92692) and chlorocebus (African green monkey, protein id: UniProtKB—P32506) was examined. FIG. 2A depicts the relative binding of all variants, which were added in saturating concentration (10 ug/ml), to NCI-H1975 cells expressing human PVR. FIG. 2B depicts the relative binding of all variants, which were added in saturating concentration (10 ug/ml), to Vero cells expressing chlorocebus PVR. For detection, Goat anti-human-647 antibody (Jackson immunoresearch 109-606-088) was used at 1:250 dilution. Cell binding of the Abs was analyzed by FACS. The fold change was calculated by dividing the MFI of each variant by the MFI of the parental antibody (K0). Significant (>25%) increase in crossreactivity is seen for N56E and N56D variants.

### Example 3—Improved NK Activation by N56E and N56T Variants

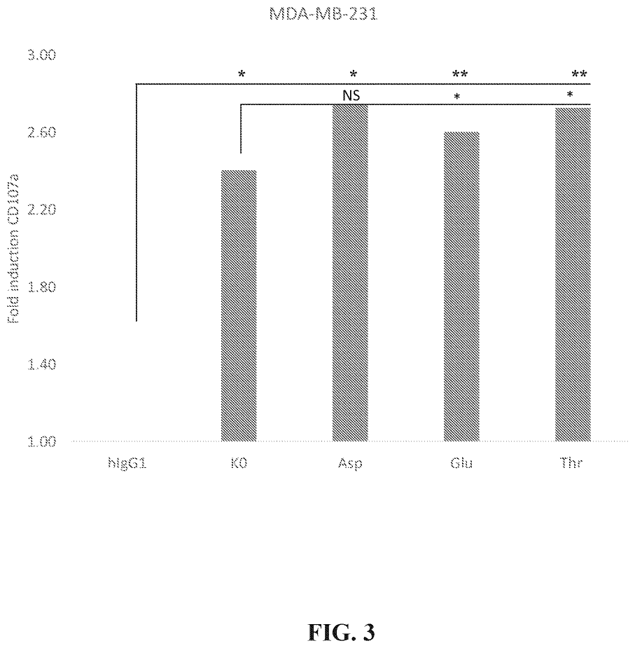

NK cells from healthy donors were incubated in presence of selected N56 substitution variants, and target breast cancer cell line (MDA-MB-231) at 2:1 E:T ratio for 2 hours at 37° C. degrees. NK cell activation was measured by the induction of surface expression of CD107a, and fold change over control IgG was calculated for each variant (Y axis). All monoclonal antibodies were used at 600 μM (0.09ug/ml). (*p<0.04, **p<0.01 by two tailed student t-test). As shown in FIG. 3, N56E and N56T variants showed improved NK activation, compared to K0, as evidenced by CD107a elevated expression

### Example 4—Improved CD8 T Cell Proliferation by N56E and N56T Variants

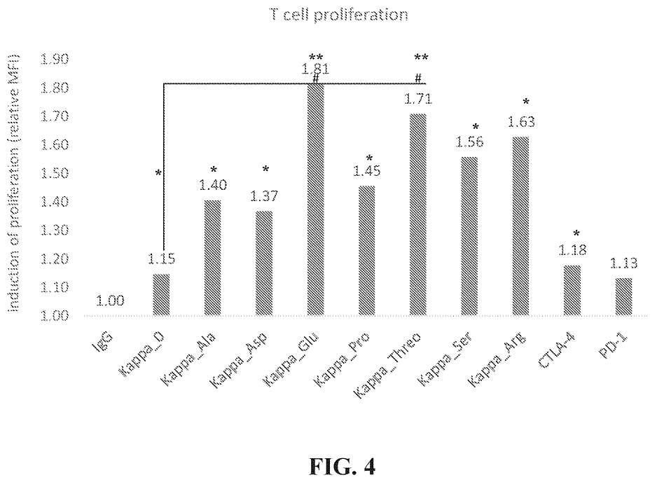

Human PBMCs were fluorescently labeled with CFSE (C34554 ThermoFischer) and incubated with A549 target breast cancer cells in the presence of 2.5ul/ml PHA-L (Roche), and with the indicated antibody variants at 4 ug/ml. After incubation for 96 hrs, the immune cells were collected, stained by anti-human CD8 and analyzed by FACS. Cell proliferation of CD8+ T cells was assessed by CFSE signal intensity. CFSE levels of the IgG treated cells were set as 1. Results are presented as fold increased proliferation relative to this control. As increase in proliferation results in reduces CFSE signal, the Y axis depicts the reciprocal value of this ratio. Experiments were done in quadruplicates; Shown are results for a single PBMC donor. The data suggest that variants N56E and N56T have significantly stronger effect on the proliferation of CD8+ T cells in presence of tumor cells compared to the parental antibody (FIG. 4; *p<0.05, **p<0.01 by two tailed student t-test).

### Example 5—Identification of a Humanized 5B9 Variant Having Improved Producibility

N56E antibody variant performed the best in the competition assay and was selected as the lead variant for humanization. Based on structural analysis, a large preliminary set of sequence segments were identified that were used to create the 5B9 humanized variants. These segments were selected and analyzed using iTope™ technology for in silico analysis of peptide binding to human MHC class II alleles (Perry et al 2008), and using the TCED™ of known antibody sequence-related T cell epitopes (Bryson et al 2010). Sequence segments that were identified as significant non-human germline binders to human MHC class II or that scored significant hits against the TCED™ were discarded. This resulted in a reduced set of segments, and combinations of these were further analyzed, as described above, to ensure that the junctions between segments did not contain potential T cell epitopes. Selected sequence segments were assembled into complete V region sequences that were devoid of significant T cell epitopes. Five heavy chain (VH1 to VH5) and 4 light chains (containing the N56E substitution) (VK1 to VK4) sequences were then chosen.

TABLE 1
variable regions and CDR sequences

| SEQ | | |
|---|---|---|
| ID | | |
| NO: | Sequence | Chain |
| 1 | QVQLVQSGAEVKKPGASVKVSCKATGYTFSNYWIEWVRQAPGQGLE | VH4 |
| | WIGEIFPGSGRINFNEKFKGRVTFTADTSISTTYMELSRLRSDDTAVYYC | |
| | ARTKIYGNSFDYWGQGTLVTVSS | |
| 2 | DIQMTQSPSSLSASVGDRVTITCKASQDVGTAVVWYQQKPGKAPKLLI | VK2 |
| | YWASSRHEGVPDRFSGSGSGTDFTLTISSLQPEDFADYFCQQYSRYPLT | |
| | FGQGTKLEIK | |
| 3 | QVQLVQSGAELKKPGASVKISCKATGYTFSNYWIEWIKQAPGQGLEWI | VH1 |
| | GEIFPGSGRINFNEKFKGR | |
| | ATFTADTSIDTTYMQLSSLTSDDSAVYYCARTKIYGNSFDYWGQGTTV | |
| | TVSS | |
| 4 | QVQLVQSGAEVKKPGASVKISCKATGYTFSNYWIEWIKQAPGQGLEWI | VH2 |
| | GEIFPGSGRINFNEKFKGRATFTADTSIDTTYMELSRLRSDDTAVYYCA | |
| | RTKIYGNSFDYWGQGTLVTVSS | |
| 5 | QVQLVQSGAEVKKPGASVKVSCKATGYTFSNYWIEWIKQAPGQGLEW | VH3 |
| | IGEIFPGSGRINFNEKFKGRVTFTADTSISTTYMELSRLRS | |
| | DDTAVYYCARTKIYGNSFDYWGQGTLVTVSS | |
| 6 | QVQLVQSGAEVKKPGASVKVSCKATGYTFSNYWIEWVRQAPGQGLE | VH5 |
| | WMGEIFPGSGRINFNEKFKGRVTFTADTSISTAYMELSRLRSDDTAVYY | |
| | CARTKIYGNSFDYWGQGTLVTVSS | |
| 7 | DIMMTQSPSFLSASVGDRVTITCKASQDVGTAVVWYQQKPGKAPKLLI | VK1 |
| | YWASSRHEGVPDRFTGSGSGTDFTLTISSLQSEDFADYFCQQYSRYPLT | |
| | FGQGTKLEIK | |
| 8 | DIQMTQSPSSLSASVGDRVTITCRASQDVGTAVVWYQQKPGKAPKLLI | VK3 |
| | YWASSRHEGVPSRFSGSGSGTDFTLTISSLQPEDFATYFCQQYSRYPLTF | |
| | GQGTKLEIK | |
| 9 | DIQMTQSPSSLSASVGDRVTITCRASQDVGTAVAWYQQKPGKAPKSLI | VK4 |
| | YWASSRHEGVPSRFSGSGSGTDFTLTISSLQPEDFATYFCQQYSRYPLTF | |
| | GQGTKLEIK | |
| 10 | NYWIE | HCCDR1 |
| 11 | EIFPGSGRINFNEKFKG | HCCDR2 |
| 12 | TKIYGNSFDY | HCCDR3 |
| 13 | X₁ASQDVGTAVX₂ X₁ = K or R, X₂ = V/A | LCCDR1 |
| 14 | WASSRHE | LCCDR2 |
| 15 | QQYSRYPLT | LCCDR3 |
| 16 | QVQLQQSGAELMKPGASVKISCKATGYTFSNYWIEWIKQRPGHGLEWI | VH0 5B9 |
| | GEIFPGSGRINFNEKFKGKATFTADTSSDTTYMQLSSLTSADSAVYYCA | |
| | RTKIYGNSFDYWGQGTTLTVSP | |
| 17 | DIMMTQSHKFMSTSVGDRVNITCKASQDVGTAVVWYQQKPGQSPKLL | VK0 5B9 |
| | IYWASSRHNGVPDRFTGSGSGTDFTLTISNVQSEDLSDYFCQQYSRYPL | |
| | TFGAGTKLELK | |

TABLE 2
Framework (Non-CDR) sequences of the humanized heavy chain
variable regions.

| Chain | FR-H1 | FR-H2 | FR-H3 | FR-H4 |
|---|---|---|---|---|
| VH 4 | QVQLVQSGAEVKKPGASV | WVRQAPGQGLEW | RVTFTADTSISTTYME | WGQGTLVTV |
| | KVSCKATGYTFS (SEQ ID | IG (SEQ ID | LSRLRSDDTAVYYCA | SS (SEQ ID |
| | NO: 18) | NO: 19) | R (SEQ ID NO: 20) | NO: 21) |
| VH 1 | QVQLVQSGAELKKPGASV | WIKQAPGQGLEWI | RATFTADTSIDTTYM | WGQGTTVTV |
| | KISCKATGYTFS (SEQ ID | G (SEQ ID | QLSSLTSDDSAVYYC | SS (SEQ ID |
| | NO: 22) | NO: 23) | AR (SEQ ID NO: 24) | NO: 25) |
| VH 2 | QVQLVQSGAEVKKPGASV | WIKQAPGQGLEWI | RATFTADTSIDTTYM | WGQGTLVTV |
| | KISCKATGYTFS (SEQ ID | G (SEQ ID | ELSRLRSDDTAVYYC | SS (SEQ ID |
| | NO: 26) | NO: 23) | AR (SEQ ID NO: 27) | NO: 21) |
| VH 3 | QVQLVQSGAEVKKPGASV | WIKQAPGQGLEWI | RVTFTADTSISTTYME | WGQGTLVTV |
| | KVSCKATGYTFS (SEQ ID | G (SEQ ID | LSRLRSDDTAVYYCA | SS (SEQ ID |
| | NO: 18) | NO: 23) | R (SEQ ID NO: 20) | NO: 21) |
| VH 5 | QVQLVQSGAEVKKPGASV | WVRQAPGQGLEW | RVTFTADTSISTAYME | WGQGTLVTV |
| | KVSCKATGYTFS (SEQ ID | MG (SEQ ID | LSRLRSDDTAVYYCA | SS (SEQ ID |
| | NO: 18) | NO: 28) | R (SEQ ID NO: 29) | NO: 21) |

TABLE 3
Framework (Non-CDR) sequences of the humanized light variable regions.

| Chain | FR-L1 | FR-L2 | FR-L3 | FR-L4 |
|---|---|---|---|---|
| LK 2 | DIQMTQSPSSLSASVGDR | WYQQKPGKAPKLLIY | GVPDRFSGSGSGTDF | FGQGTKLEI |
| | VTITC (SEQ ID NO: 30) | (SEQ ID NO: 31) | TLTISSLQPEDFADYF | K (SEQ ID |
| | | | C (SEQ ID NO: 32) | NO: 33) |
| LK 1 | DIMMTQSPSFLSASVGDR | WYQQKPGKAPKLLIY | GVPDRFTGSGSGTDF | FGQGTKLEI |
| | VTITC (SEQ ID NO: 34) | (SEQ ID NO: 31) | TLTISSLQSEDFADYF | K (SEQ ID |
| | | | C (SEQ ID NO: 35) | NO: 33) |
| LK 3 | DIQMTQSPSSLSASVGDR | WYQQKPGKAPKLLIY | GVPSRFSGSGSGTDFT | FGQGTKLEI |
| | VTITC (SEQ ID NO: 30) | (SEQ ID NO: 31) | LTISSLQPEDFATYFC | K (SEQ ID |
| | | | (SEQ ID NO: 36) | NO: 33) |
| LK 4 | DIQMTQSPSSLSASVGDR | WYQQKPGKAPKSLIY | GVPSRFSGSGSGTDFT | FGQGTKLEI |
| | VTITC (SEQ ID NO: 30) | (SEQ ID NO: 37) | LTISSLQPEDFATYFC | K (SEQ ID |
| | | | (SEQ ID NO: 36) | NO: 33) |

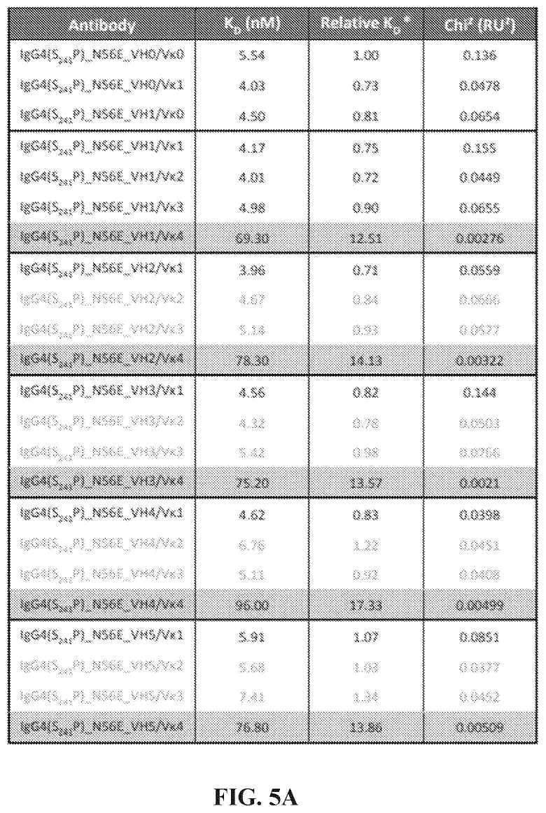

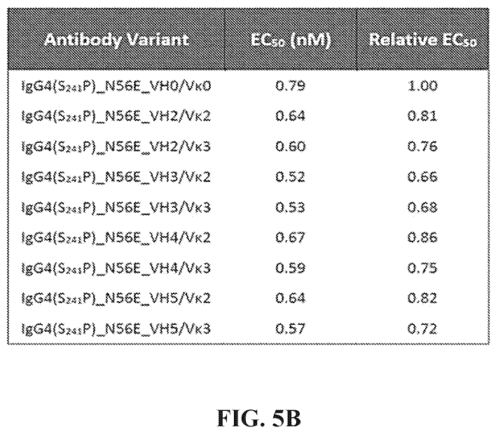

All variants were tested for binding by SPR (FIG. 5A) and variants with affinity of 2× from the parental antibody were tested for cell-surface PVR binding by flow cytometry (FIG. 5B). With the exception of variants containing Vk4, all variants showed very similar binding compared to the parental mouse/human chimeric molecule carrying the N56E substitution (IgG4(S241P) N56E_VH0/VK0). It is noted that the humanization removed an N-Linked glycosylation at position N20 FR1 light chain.

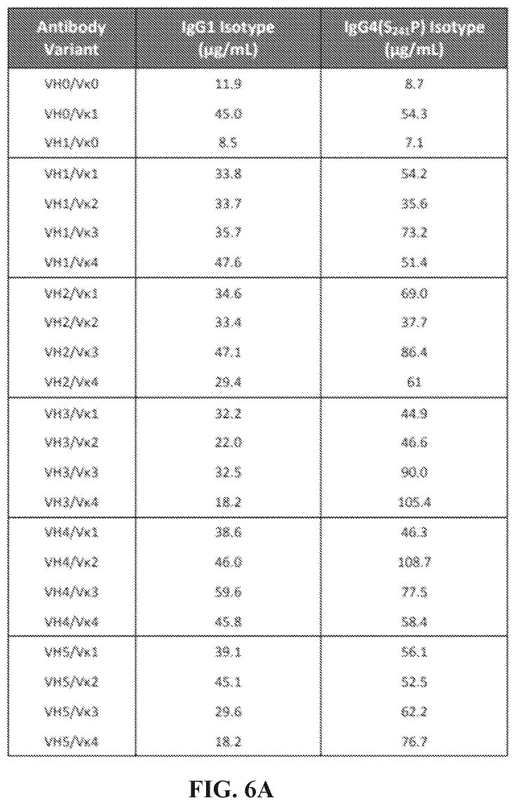

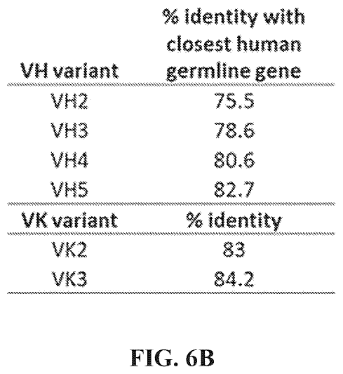

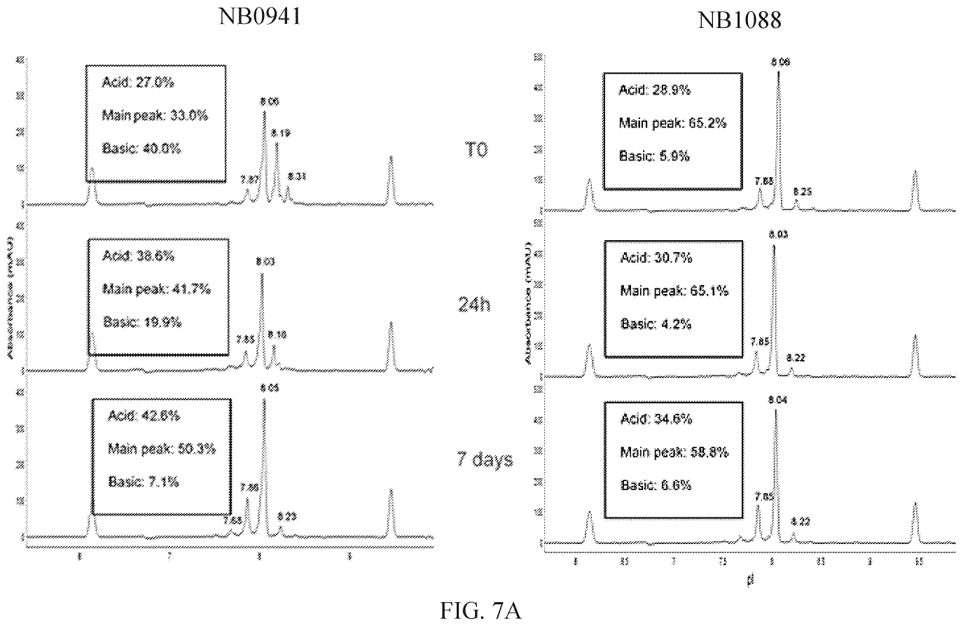

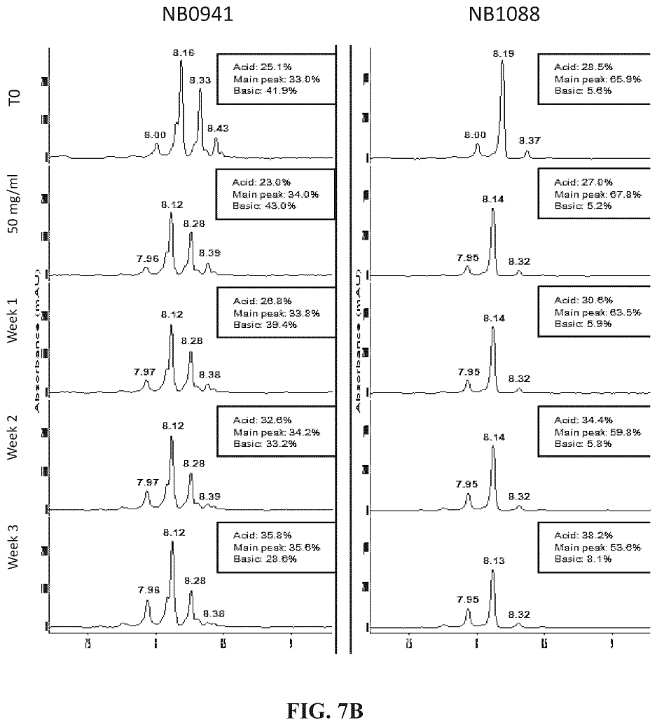

To select a lead candidate, the expression levels after transient expression in HEK 293 EBNA cells, and similarity to human germline sequence were considered (FIG. 6). FIG. 6A summarizes the titers of all variants after transient transfection. Variant VH4/Vk2 showed the highest expression titers and possesses a high percentage of sequence identity with human germ line genes (FIG. 6B). Finally, a producibility assessment of VH4/Vk2 variant (NB 1088) was performed in comparison to a variant identical to NB 1088, but with the original, deamidation competent LC CDR2 of 51B9 (WASSRHNG) termed N1B0941. The biophysical properties of NB0941 and NB 1088 were determined. As shown in FIGS. 7A and 7B, high pH stress and incubation at 40° C. revealed changes in the capillary isoelectric focusing (cIEF), specifically an increase in the percent of acid species, possibly due to deamidation. These changes were more pronounced in NB0941 compared to NB1088. Therefore, NB1088 with optimized immunogenicity, expression and binding profile as well as desirable biophysical properties was chosen as the lead humanized variant for functional analysis.

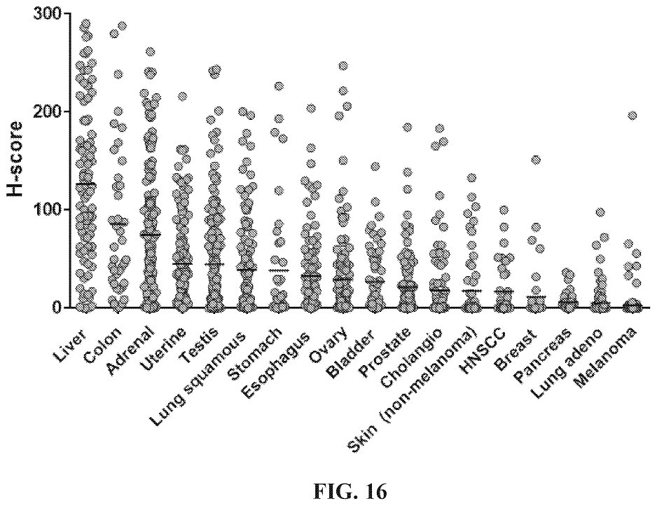

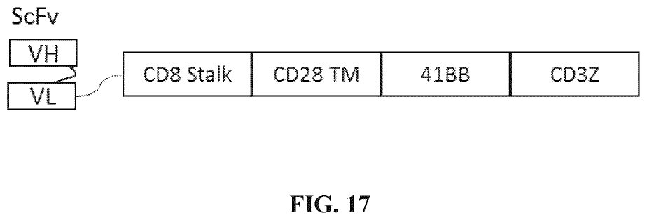

The decrease in affinity, observed in some of the variants, is particularly advantageous in designing CAR driver, given the fact that normal tissues express PVR at minimal levels. The results (FIG. 16) indicate that PVR is overexpressed in variety of tumors, allowing for efficient targeting of these tumors by PVR driven CAR-T. The potential safety concern can be easily addressed by the affinity “tuned down” anti-PVR variants as described in Liu et al. (Cancer research, 2015; Volume 75, Issue 17).

### Example 6—NB1088 Inhibits Binding of PVR to TIGIT, CD96 and CD226

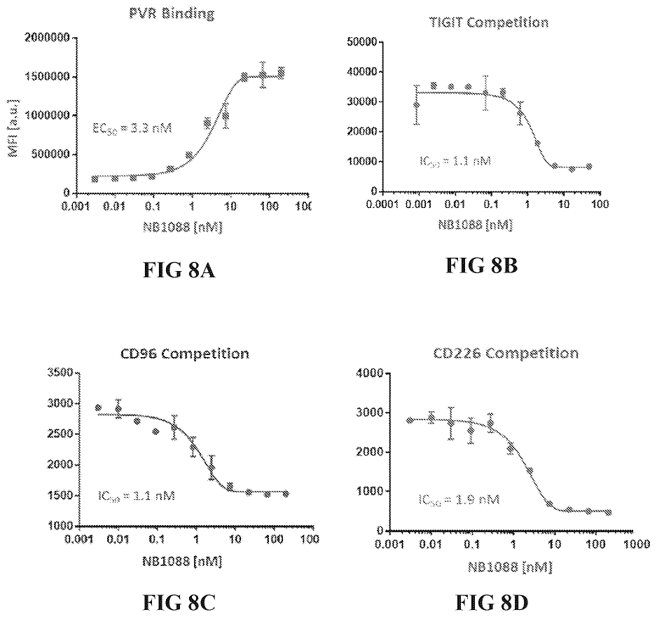

NB1088 was tested for its ability to block TIGIT, CD96, and CD226 binding to PVR. Dissociated CHO cells (Chinese Hamster Ovary) stably expressing human PVR were incubated with NB1088 at the indicated concentrations for 20 minutes on ice followed by addition of biotinylated recombinant TIGIT, CD96 or CD226-Fc, respectively, at 10ug/ml for an additional 120 minutes on ice. After washing, surface bound NB1088 was detected with anti-human Alexa-488 conjugated secondary antibody and biotinylated proteins were detected with Alexa647 conjugated Streptavidin and analyzed by flow cytometry. FIG. 8A shows that NB1088 binds to PVR with an EC₅₀ of about 3.3 nanomolar. The IC₅₀ for NB1088 to compete with TIGIT, CD96, or CD226 for PVR binding is 1.1, 1.1 and 1.9 nM respectively, as shown in FIGS. 8B to 8D.

### Example 7—NB1088 Stimulates Cytotoxic T and NK Cells

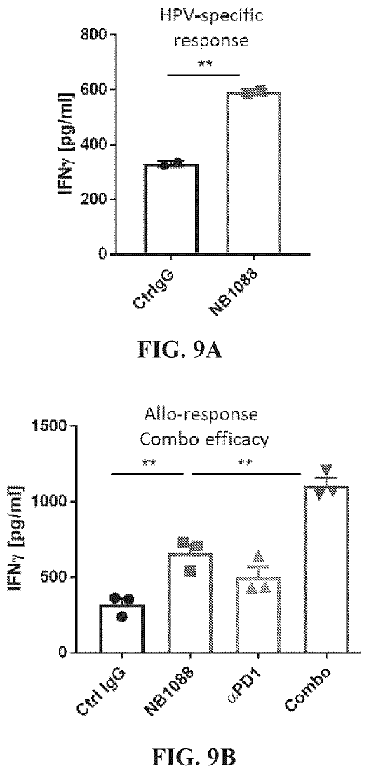

The ability of NB1088 to stimulate T and NK cell activity in vitro was determined. Using an antigen-specific human papillomavirus (HPV) assay, 30,000 HPV+ human cervical epidermoid carcinoma cell line (CaSki cells) and 30,000 HPV-specific CD8 T-cells were co-incubated with control IgG or NB1088 at 10 ug/ml overnight. Interferon gamma release into the supernatant was detected using a human interferon gamma specific MSD system. As shown in FIG. 9A, NB1088 increased the interferon gamma release from human, HPV-specific CD8+ (cytotoxic) T cells when incubated with HPV+ CaSki cells. To test CD8 T-cell activity in an allogenic system, PBMCs were preactivated for three days with Phytohemagglutinin (PHA) and Interleukin 2 (IL2), rested overnight in the absence of PHA/IL2 before isolating CD8 T-cells using a magnetic, negative isolation procedure. 10,000 A549 tumor cells and 100,000 healthy donor CD8+ T cells were co-cultured overnight in the presence of 100 U/ml IL2 and lug/ml anti-CD28 antibody. As shown in FIG. 9B, NB1088 increased interferon gamma release from CD8+ T cells to a greater extent than anti-PD-1 (pembrolizumab), which was further increased as a result of the combination of the NB1088 and the anti-PD-1 antibody.

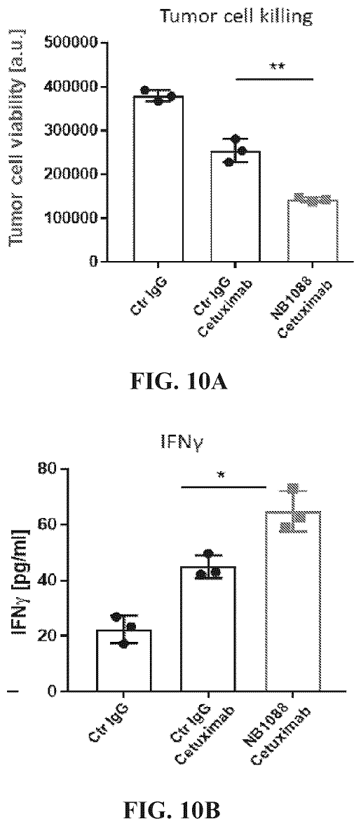

The effect of NB1088 on antibody dependent cell cytotoxicity (ADCC) was also determined. NK cells from healthy donors were isolated from PBMCs rested overnight using a magnetic, negative isolation procedure. 10,000 PVR+ and EGFR+ A549 tumor cells and 50,000 NK cells were either incubated with control IgG; control IgG and the anti-EGFR antibody cetuximab (5 ug/mL); or cetuximab and NB1088. Activity of NK cells to mediate antibody dependent cytotoxicity or interferon gamma release was determined with Cell Titer Glow by analyzing viability of adherent A549 after co-culture and removal of NK-cells, or by MSD analysis of supernatants as above. As shown in FIG. 10A and FIG. 10B, NB1088 when incubated with cetuximab was able to increase the NK-cell mediated killing of A549 cells as well as interferon gamma release.

### Example 8—NB1088 Restores CD226 Expression and Activity on CD8 T and NK Cells

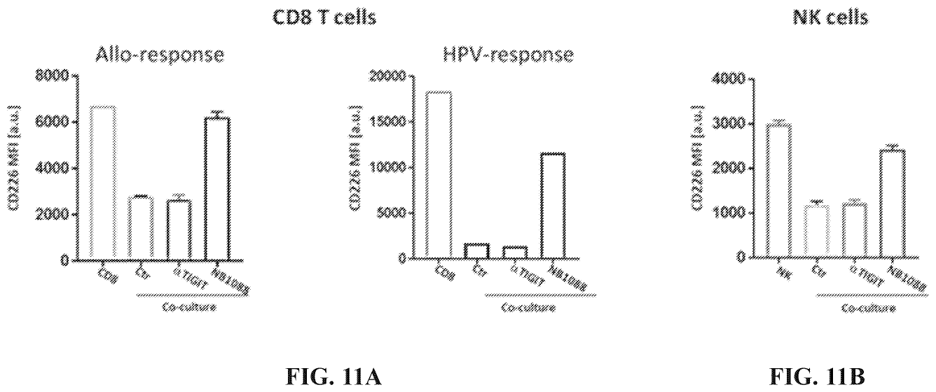

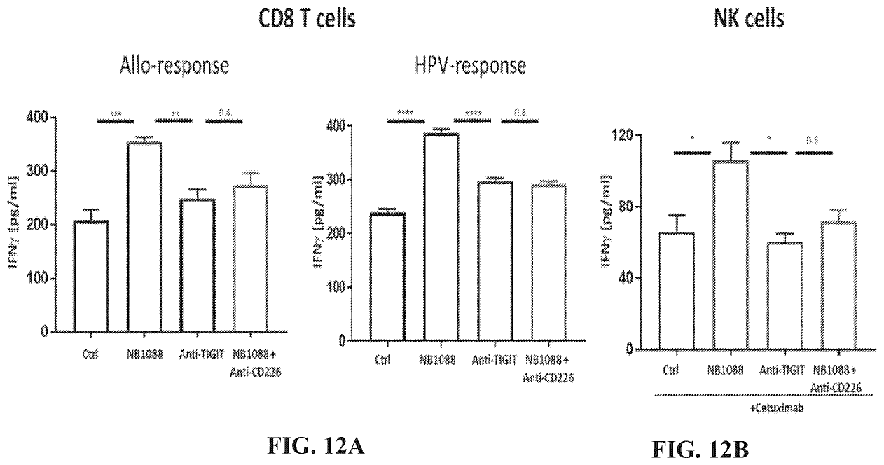

The ability of NB1088 to affect the function of CD226 was determined. CD226 (DNAM-1) is a cell-surface glycoprotein receptor, expressed by NK and T cells, that serves as a ligand for PVR and aides in tumor killing by CD8+ T and NK cells. In its function, it is opposed by TIGIT and CD96, which are inhibitory molecules expressed on T and NK cells. Thus, an increase in CD226 function due to NB1088 would indicate that NB1088 would enhance T and NK cell activity and have broad anti-tumor activity. The impact of NB1088 on CD226 expression and function was tested in antigen specific and allogenic co-culture systems as described above. As shown in FIGS. 11A and 11B, co-culture of CD8 T cells and NK cells with PVR+ target cells led to a strong reduction in CD226 surface expression on CD8 T cells and NK cells. NB1088 restored cell surface expression of CD226 on CD8 T cells or NK cells, regardless of co-culture system (FIGS. 11A and B) whereas anti-TIGIT did not. The functional consequence of increased CD226 expression on T and NK cells following NB1088 treatment was evaluated using the antigen specific and allogenic coculture systems described above with slight modifications (FIGS. 12A and B). Increased CD226 expression correlated with significantly higher levels of interferon gamma release following NB1088 treatment compared to control IgG or anti-TIGIT treatment (FIGS. 12A and B). Superior T and NK cell activity with NB1088 treatment was at least partially mediated via CD226 activity. Anti-CD226 (DX11, 20ug/ml) reduced NB1088-dependent interferon gamma release by both allo- and antigen-stimulated CD8 T cells (FIG. 12A) and NK cells following A549 co-culture (FIG. 12B) to levels observed with anti-TIGIT. These data demonstrate that NB1088 improves T and NK cell activity over TIGIT blockade by increasing CD226 expression and/or function.

### Example 9—NB1088 Monotherapy Efficacy in Humanized Mouse Tumor Xenograft Models

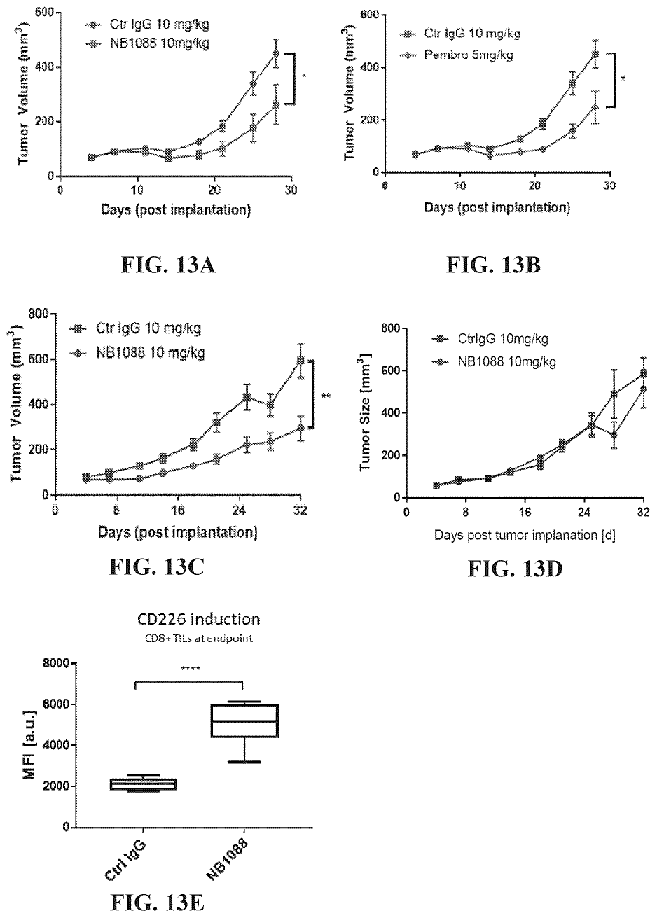

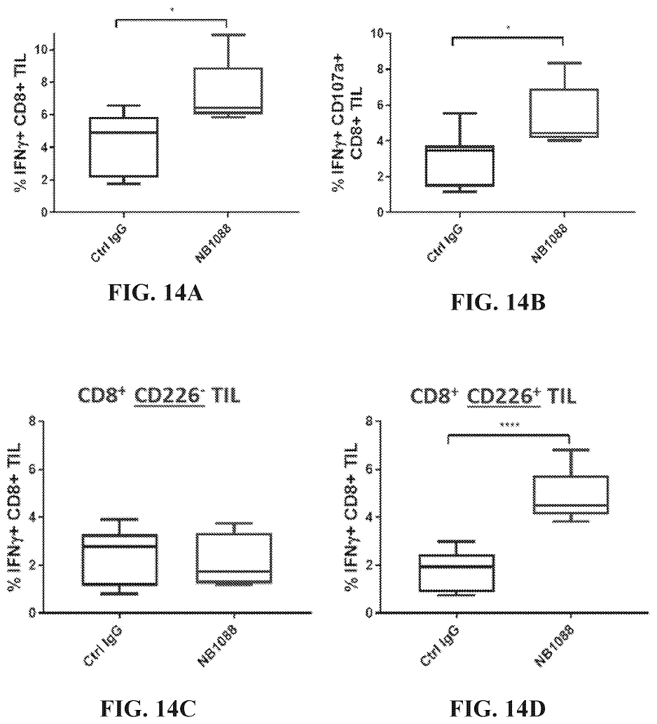

The ability of NB1088 to kill tumors in a humanized mouse model, either A549 (lung adenocarcinoma) or HPAF (pancreatic) was determined. Briefly, 5×10⁶ tumor cells (either A549 or HPAF) were mixed with activated human peripheral blood mononuclear cells at a 1:1 ratio in matrigel and implanted subcutaneously into the flank of immunodeficient NOD/SCID mice (12 animals per condition). As shown in FIGS. 13A and 13B, NB1088 was able to reduce tumor volume at least as well as the anti-PD-1 antibody pembrolizumab. NB1088 was also able to reduce tumor volume in the A549/PBMC model (FIG. 13C), but not with A549 cells alone (FIG. 13D). In the A549/PBMC model, reduced tumor volume correlated with increased CD226 expression on CD8 T cells isolated from NB1088-treated tumors. (FIG. 13E). The effects of NB1088 on CD8 T cell effector function ex vivo was also assessed (FIGS. 14A and B). Digested single cell suspensions from tumors were stimulated with anti-CD28/anti-CD3 in the presence of Brefeldin A and anti-CD107a for 5 hrs at 37° C. Following stimulation, cells were stained to detect production of interferon gamma by CD8+ T cells using flow cytometry by standard surface/intracellular staining methods. As shown in FIGS. 14A and 14B, NB1088 increased the frequency of total interferon gamma positive (FIG. 14A) and polyfunctional interferon gamma/CD107a double positive (FIG. 14B) tumor derived CD8 T-cells. Furthermore, the increased frequency of interferon gamma positive CD8+ T cells in NB1088 treated tumors were derived exclusively from CD226 positive CD8+ T-cells (FIGS. 14C and 14D), suggesting an important in vivo contribution of CD226 function to the anti-tumor activity of NB1088.

### Example 10—NB1088 Pharmacokinetics and Pharmacodynamic Changes in CD226 Expression on CD4 T Cells in Cynomolgus Monkey

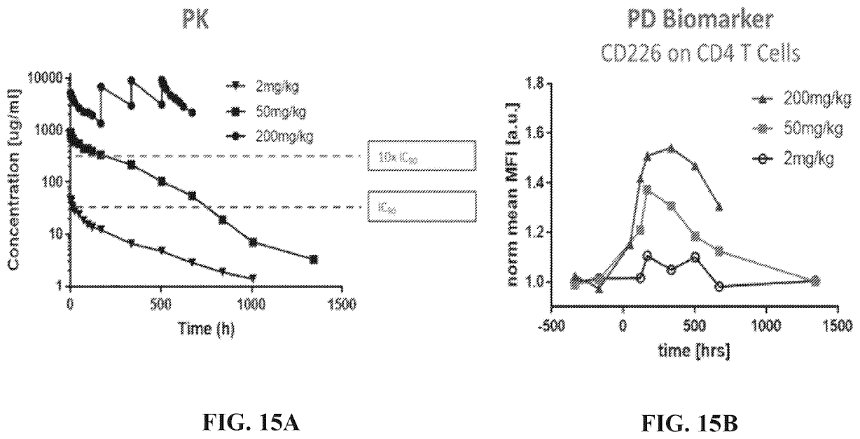

The pharmacokinetic properties of NB1088 was measured following a single or 4× 1 weekly IV bolus injections at 2, 50 or 200 mg/kg dose levels in cynomolgus monkey (2 female monkeys/dose group). In addition, changes in CD226 expression on circulating peripheral CD4 T cells was evaluated. FIG. 15A shows the plasma concentrations (ug/ml) of NB1088 as a function of time (hours) and dose. IC90 and 1Ox IC90 were calculated based on in vitro potency assays using cynomolgus monkey PBMC assay. NB1088 shows a typical PK profile and reached concentrations above 10x IC90 for the duration of the study following repeat dosing at 200 mg/kg dose level. FIG. 15B shows CD226 expression levels on circulating CD4 T-cells normalized to pre-dose, as measured by flow cytometry with specific antibodies. NB1088 increased CD226 surface expression levels up to 1.5-fold in the 50 mg/kg dose group and 200 mg/kg repeat dose group and remained elevated in the 200 mg/kg repeat dose group. These data indicate that NB1088 can engage and modulate CD226 expression on CD4 T cells in cynomolgus monkey.

### Example 11—Expression of Human PVR Across Different Tumor Types

The expression levels of PVR in human cancer of different origin was evaluated. PVR expression was detected by standard immunohistochemistry procedures using the commercially available rabbit monoclonal antibody clone D3G7H and cancer tissue microarrays. Staining was digitized and intensities were quantified to calculate H-scores within and across indications. FIG. 16 shows elevated expression levels of PVR in most indications analyzed at varying frequencies. The elevated expression of PVR was shown in liver cancer, colon cancer, adrenal cancer, uterine cancer, testicular cancer, squamous cell lung cancer, stomach cancer, esophagus cancer, ovary cancer, bladder cancer, prostate cancer, Cholangiocarcinoma, skin cancer, HNSCC cancer, breast cancer, pancreatic cancer, non-small cell lung cancer, and melanoma. These data suggest contribution of PVR to tumor progression in multiple indications of human cancer.

### Example 12—Designing a Humanized Antibody

Humanized IgG antibodies were designed based on one of the variants having the heavy and light chains V_{H}4 and VK2, respectively. An exemplified VK2 sequence is set forth in SEQ ID NO: 49. Exemplified V_{H}4 sequences for hIgG4 (S241P) is set forth in SEQ ID NO: 50, and for hIgG1 is set forth in SEQ ID NO: 51. Further, exemplary nucleotide sequences optimized for expressing the amino acid sequences in CHO cells were designed as follows: For VK2, nucleotide sequence set forth in SEQ ID NO: 52 or SEQ ID NO: 53. For V_{H}4 of IgG4 nucleotide sequence set forth in SEQ ID NO: 54 or SEQ ID NO: 55.

### Example 12—CAR-T Cells Expressing scFv Derived from Humanized Anti-PVR Antibody Variants are Specifically Activated in the Presence of Tumor Cells

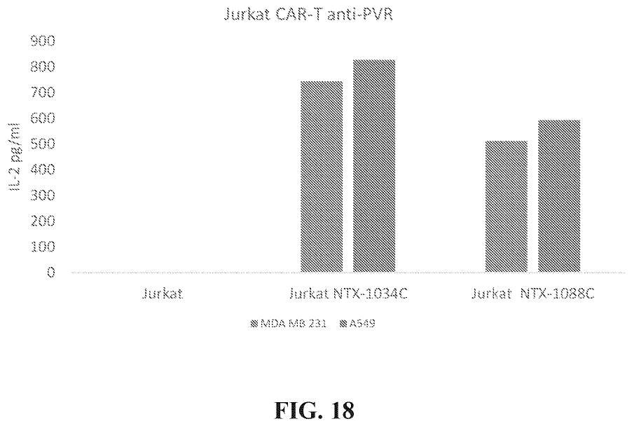

CAR-T construct were designed based on variants H4K2-NTX-1088C and H3K4-NTX-1034C. The amino acid sequences of the scFv molecules are set forth in SEQ ID Nos: 56 and 57, respectively. Parental Jurkat cells or Jurkat cells overexpressing anti-hPVR CAR-T (40K/well) were incubated with A549 or MDA-231 breast cancer cells (PVR positive) at 1:1 E:T for 24 hours. As shown in FIG. 18, both CAR-T drivers led to secretion of hundreds pg of IL2, while the parental Jurkat cells had no detectable IL2 secreted, in presence of the indicated targets. IL2 secretion was quantified using Biolegend hIL2 (cat 431804) These results suggest that uPVR based CAR-T driver is highly functional in inducing T cells activation in presence of target cells expressing PVR.

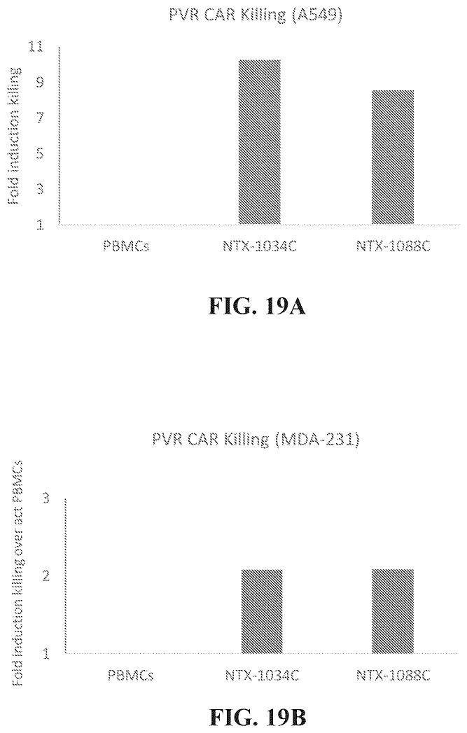

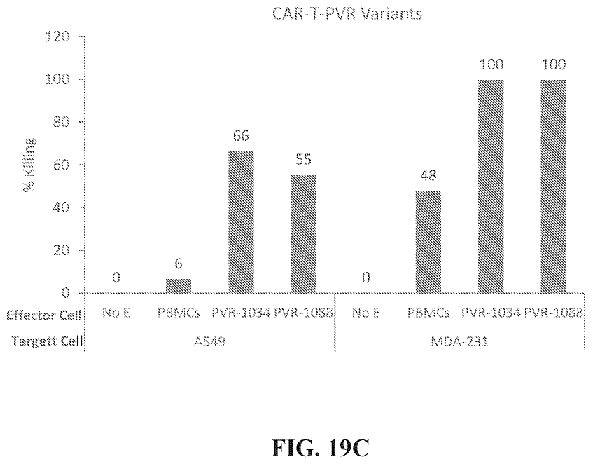

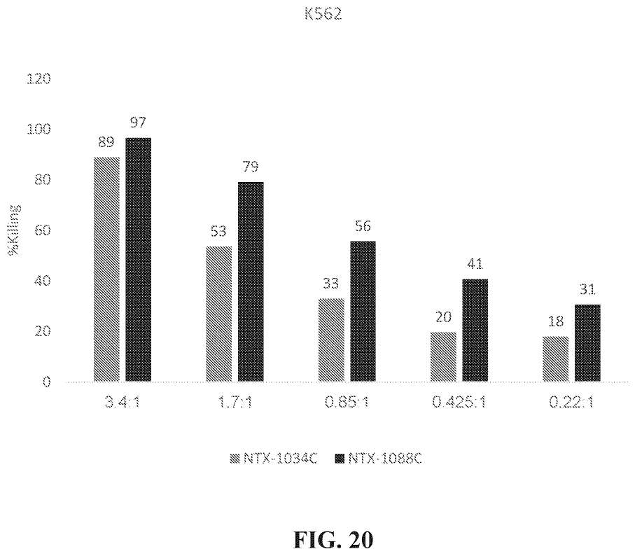

To examine the CAR-T tumor cell killing, 200K of A549 or MDA-231 cells were plated in a plate of 12 wells with either CAR-T-PVR variants (NTX-1088C or NTX1034C) at E:T of 0.4 and 0.8 to 1 (based on GFP positivity) respectively in NK media for 72 hours. Tumor cell killing was assessed using the standard CTG protocol (Promega G9241). As shown in FIGS. 19A-19C, both PVR variants exhibited over 2-fold increased killing of MDA-231 cells and 8-fold increased killing of A549 cells compared to activated PBMCs. These findings strongly suggest that the uPVR CAR-T constructs significantly increase the killing of targets expressing PVR.

### Example 13—Efficient Hematological Target Cell Killing by αPVR CAR-Ts

CAR-T construct were designed based on variants H4K2-NTX-1088C and H3K4-NTX-1034C. The scFv sequences are set forth in SEQ ID Nos: 56 and 57, respectively.

To examine the CAR-T hematological tumor cell killing, 20K/well of K562 cells were plated in 96 wells plates either alone or with CAR-T-PVR variants (NTX-1088C or NTX1034C) at E:Ts ranging from 3.4 to 0.22 to 1 in RPMI supplemented with 100IU/IL-2/ml for 18 hours. Tumor cell killing was evaluated by flow cytometry. Both NTX-1034C and NTX-1088C were extremely effective in eliminating the targets at higher E:Ts. A clear advantage in NTX-1088C over NTX-1034C at lower E:Ts is probably due to the moderate levels of PVR expressed on K562. These results suggest that uPVR CAR-T can be effective against hematological tumors that express PVR.

We claim:

1. A method of treating a cancer in an individual afflicted with a cancer comprising administering to the individual a therapeutically effective amount of: (i) a humanized antibody or antigen binding fragment thereof comprising heavy chain variable region comprises an amino acid sequence set forth in SEQ ID NO: 47, and the light chain variable region comprises an amino acid sequence set forth in SEQ ID NO: 48; and (ii) an additional anti-cancer agent.

2. The method of claim 1, wherein the anti-cancer agent is an agent that downregulates the activity or expression of an immune co-inhibitory receptor.

3. The method of claim 2, wherein the immune co-inhibitory receptor is selected from the group consisting of PD-1, PD-L1, TIGIT, CTLA-4, LAG3, TIM3, BTLA, VISTA, B7H4, CD96, BY55 (CD 160), LAIR1, SIGLEC10, CD112R, CD112, ILT-4 and 2B4.

4. The method of claim 1, wherein the anti-cancer agent is an inhibitor of PD-1, PD-L1, CTLA-4 or CD112R signaling.

5. The method of claim 4, wherein the anti-cancer agent is an inhibitor of PD-1 signaling.

6. The method of claim 5, wherein the agent is an antibody or fragment thereof that binds to PD-1.

7. The method of claim 6, wherein the antibody or fragment thereof that binds to PD-1 is selected from the group consisting of Pembrolizumab, Nivolumab, AMP-514, Tislelizumab, Spartalizumab, and a binding fragment thereof.

8. The method of claim 4, wherein the agent is an antibody that specifically binds PD-L1 or PD-L2.

9. The method of claim 8, wherein the antibody that specifically binds PD-L1 or PD-L2 is selected from the group consisting of Durvalumab, Atezolizumab, Avelumab, BMS-936559, or FAZ053, and a binding fragment thereof.

10. The method of claim 1, wherein the agent is a small molecule that inhibits PD-1, PD-L1, or PD-L2.

11. The method of claim 1, wherein the agent is an anti-endothelial growth factor receptor (EGFR) antibody.

12. The method of claim 1, wherein the cancer comprises a solid tumor.

13. The method of claim 1, wherein the cancer is selected from the group consisting of glioblastoma, pancreatic cancer, breast cancer, bladder cancer, kidney cancer, head and neck cancer, ovarian cancer, gastrointestinal cancer, colon cancer, cervical cancer, prostate cancer, and lung cancer.

14. The method of claim 1, wherein the cancer is a hematological cancer.

15. The method of claim 1, wherein the antibody or fragment thereof comprising:
(1) a heavy chain variable region comprising:
i. a set of three CDR sequences comprising the sequences set forth in SEQ ID Nos. 10-12; and
ii. a set of four heavy chain framework (FR) sequences: (A) FR-H1 selected from the group consisting of SEQ ID NOs: 18, 22, and 26; (B) FR-H2 selected from the group consisting of SEQ ID NOs: 19, 23, and 28; (C) FR-H3 selected from the group consisting of SEQ ID NOs: 20, 24, 27, and 29; and (D) FR-H4 selected from the group consisting of SEQ ID NOs: 21 and 25; and
(2) a light chain variable region comprising:
i. a set of three CDR sequences comprising the sequences set forth in SEQ ID Nos. 13-15; and
ii. a set of four light chain framework sequences: (A) FR-L1 selected from the group consisting of SEQ ID NOs: 30 and 34; (B) FR-L2 selected from the group consisting of SEQ ID NOs: 31 and 37; (C) FR-L3 selected from the group consisting of SEQ ID NOs: 32, 35, and 36; and (D) FR-L4 is SEQ ID NOs: 33.

16. The method of claim 1, wherein the humanized antibody or antigen binding fragment thereof comprising a heavy chain variable region comprises an amino acid sequence at least about 95% identical to a sequence selected from the group consisting of SEQ ID NO: 1, SEQ ID NO: 3, SEQ ID NO: 4, SEQ ID NO: 5 and SEQ ID NO: 6; and a light chain variable region comprises an amino acid sequence at least about 95% identical to a sequence selected from the group consisting of SEQ ID NO: 2, SEQ ID NO: 7, SEQ ID NO: 8 and SEQ ID NO: 9.

17. The method of claim 1, wherein the humanized antibody or antigen binding fragment thereof comprising a heavy chain variable region comprises an amino acid sequence at least about 97% identical to a sequence selected from the group consisting of SEQ ID NO: 1, SEQ ID NO: 3, SEQ ID NO: 4, SEQ ID NO: 5 and SEQ ID NO: 6, and a light chain variable region comprises an amino acid sequence at least about 97% identical to a sequence selected from the group consisting of SEQ ID NO: 2, SEQ ID NO: 7, SEQ ID NO: 8 and SEQ ID NO: 9.

18. The method of claim 1, wherein the humanized antibody or antigen binding fragment thereof comprising a heavy chain variable region comprises an amino acid sequence identical to a sequence selected from the group consisting of SEQ ID NO: 1, SEQ ID NO: 3, SEQ ID NO: 4, SEQ ID NO: 5 and SEQ ID NO: 6, and wherein the light chain variable region comprises an amino acid sequence identical to a sequence selected from the group consisting of SEQ ID NO: 2, SEQ ID NO: 7, SEQ ID NO: 8 and SEQ ID NO: 9.

19. The method of claim 1, wherein the humanized antibody or antigen binding fragment thereof comprising a heavy chain variable region comprises the amino acid sequence set forth in SEQ ID NO: 1, and the light chain variable region comprises the amino acid sequence set forth in SEQ ID NO: 2.

20. A method of treating a cancer in an individual afflicted with a cancer comprising administering to the individual a therapeutically effective amount of chimeric antigen receptor (CAR) NK or T cells comprising a combination of heavy and light chain variable region sequences of a humanized antibody comprising heavy chain variable region comprises an amino acid sequence set forth in SEQ ID NO: 47, and the light chain variable region comprises an amino acid sequence set forth in SEQ ID NO: 48, in combination with a further anti-cancer agent.
